(19) United States
(12) Patent Application Publication
(10) Pub. No.: US 2024/0177868 A1
(43) Pub. Date: May 30, 2024

(54) ALTERING THE INTESTINAL MICROBIOME IN CYSTIC FIBROSIS

(71) Applicants: Trustees of Dartmouth College, Hanover, NH (US); Mary Hitchcock Memorial Hospital, for itself and on behalf of Dartmouth-Hitchcock Clinic, Lebanan, NH (US)

(72) Inventors: George A. O'Toole, Hanover, NH (US); Juliette C. Madan, Lyme, NH (US); Rebecca A Valls, Lebanon, NH (US); Thomas Hampton, Hanover, NH (US)

(21) Appl. No.: 18/521,285

(22) Filed: Nov. 28, 2023

Related U.S. Application Data
(60) Provisional application No. 63/428,417, filed on Nov. 28, 2022.

(51) Int. Cl. G16H 50/50; C12Q 1/689; G16B 40/20

(52) U.S. Cl. CPC G16H 50/50; C12Q 1/689; G16B 40/20; C12Q 2600/118

(57) ABSTRACT

The present disclosure relates to computer-implemented systems for identifying a patient with cystic fibrosis as having a high risk for upper respiratory infections or systemic inflammation, based on the patient's stool microbiota. The systems use machine learning models trained with data comprising (a) risk classifications for subjects with cystic fibrosis and (b) stool microbiota information for each of the subjects. The present disclosure also relates to methods of reducing frequency and/or number of upper respiratory infections in a patient with cystic fibrosis. The present disclosure also relates to methods of identifying a patient with cystic fibrosis (CF) as having a high risk for frequent upper respiratory infections or high systemic inflammation based on the patient's stool microbiota.

### CROSS REFERENCE TO RELATED APPLICATIONS

This patent application claims priority to U.S. Provisional Patent Application No. 63/428,417, filed on Nov. 28, 2022, the entire contents of which are fully incorporated herein by reference.

### FEDERALLY SPONSORED RESEARCH OR DEVELOPMENT

This invention was made with government support under T32 HL134598-01 and T32-AI007363 and P30-DK117469 awarded by the National Institutes of Health. The government has certain rights in the invention.

### TECHNICAL FIELD

Methods and computer systems for identifying risk of upper respiratory infections or systemic inflammation in patients having cystic fibrosis, and for diagnosing, predicting, or preventing cystic fibrosis-associated negative clinical outcomes in such patients.

### BACKGROUND

Support was also provided under grants OTOOLE19GO, 003037G221 and MADAN596389 from the Cystic Fibrosis Foundation, STANTO19R0 from the Cystic Fibrosis Foundation Research Development Program, and from the Innovation PhD Program at Dartmouth.

Cystic fibrosis (CF) is a genetic disease associated with chronic lung infections that lead to overall decline in lung health (see Price C E, O'Toole G A. The gut-lung axis in cystic Fibrosis. J Bacteriol. 2021; 203(20); De Lisle R C, Borowitz D. The cystic fibrosis intestine. Cold Spring Harb Perspect Med. 2013; 3(9):1-17). These chronic lung infections begin in early childhood and are often preceded by pulmonary exacerbation event(s). Pulmonary exacerbations in CF are non-standardized events most commonly described as acute inflammatory responses to infection that lead to a sharp decrease in lung function measured by FEV1 (forced expiratory volume in 1 second) (Price et al., 2021). Although early research focused on the microbial communities in the lungs of children with CF (cwCF), recent research has shown connections between CF and changes in intestinal microbial communities, including in children. (Nielsen S, Needham B, Leach S T, et al. Disrupted progression of the intestinal microbiota with age in children with cystic fibrosis. Sci Rep. 2016; 6 (October 2015): 1-11; Hoffman L R, Pope C E, Hayden H S, et al. Escherichia coli dysbiosis correlates with gastrointestinal dysfunction in children with cystic fibrosis. Clin Infect Dis. 2014; 58(3):396-399; Ooi C Y, Syed S A, Rossi L, et al. Impact of CFTR modulation with Ivacaftor on gut microbiota and intestinal inflammation. Sci Rep. 2018; 8(1): 1-8; Kristensen M, Prevaes S M P J, Kalkman G, et al. Development of the gut microbiota in early life: The impact of cystic fibrosis and antibiotic treatment. J Cyst Fibros. 2020; 19(4):553-561; Antosca K M, Chernikova D A, Price C E, et al. Altered gut microbiota and intestinal inflammation. J Bacteriol. 2019; 201(16):1-15; Hayden H S, Eng A, Pope C E, et al. Fecal dysbiosis in infants with cystic fibrosis is associated with early linear growth failure. Nat Med. 2020; 26(2):215-221). Such observations are not surprising, because CF alters the intestinal environment the microbial communities inhabit, affecting the microbes' ability to establish themselves and flourish (Price et al., 2021; De Lisle et al., 2013; Kristensen et al., 2020; Meeker S M, Mears K S, Sangwan N, et al. CFTR dysregulation drives active selection of the gut microbiome. PLOS Pathog. 2020; 16(1):1-17). Intestinal microbial communities are further modulated by the treatments that cwCF regularly undergo, such as: high calorie diet, frequent use of antimicrobial agents, pancreatic insufficiency, requirement for enzyme replacement, and most recently, CFTR modulators (Nielsen et al., 2015; Ooi et al., 2018; Antosca et al., 2019). CFTR modulators like Ivacaftor not only improve lung function, but have recently been shown to return the CF intestinal environment to a more nonCF-like state by increasing pH and reducing viscosity of secreted mucin (Ooi et al., 2018). Additionally, changes in microbial communities are associated with differences in local and systemic markers of inflammation (Yoon H Y, Kim H N, Lee S H, et al. Association between neutrophil-to-lymphocyte ratio and gut microbiota in a large population: a retrospective cross-sectional study. Sci Rep. 2018; 8(1): 1-9; Hoffman et al., 2014).

Previously reported changes in the gut microbiota in cwCF include reduced Bifidobacterium and Bacteroides, with an increase in Escherichia-Shigella and Clostridium—although these shifts can depend on the cohort being examined (see Price et al., 2021; Hoffman et al., 2014; Antosca et al., 2019). Alpha diversity in cwCF increases as children age but is consistently lower than infants without CF (Price et al., 2021; De Lisle et al., 2013). The changes in microbial communities outlined here are also likely associated with alterations in lung health outcomes. (Hoen A G, Li J, Moulton L A, et al. Associations between gut microbial colonization in early life and respiratory outcomes in cystic fibrosis. J Pediatr. 2015; 167(1): 138-147.e3). The communication between the microbial communities in the gut and the lungs is referred to as the “gut-lung axis”, whereby microbes in the gut stimulate both local and distal immune responses through their secretion of metabolites and other microbial factors (Price et al., 2021). This interaction can be positive, for example by downregulating inflammation through the secretion of short chain fatty acids by these microbes, including butyrate, which can travel systemically and engage with free fatty acid receptors in the lungs (Yip W, Hughes M R, Li Y, et al. Butyrate shapes immune cell fate and function in allergic asthma. Front Immunol. 2021; 12(February):1-13). Additionally, Bacteroides-secreted glycolipids can activate colonic dendritic cells that induce local and systemic cytokine release, which in turn enhance resistance to pulmonary viral infection (Stefan K L, Kim M V., Iwasaki A, Kasper D L. Commensal microbiota modulation of natural resistance to virus infection. Cell. 2020; 183(5): 1312-1324.e10). Commensal derived metabolites, such as riboflavin, can also imprint the abundance of mucosal-associated invariant T cells (Constantinides M G, Link V M, Tamoutounour S, et al. MAIT cells are imprinted by the microbiota in early life and promote tissue repair. Science (80-). 2019; 366(6464)). The interaction between gut microbiota and the host can also be negative, whereby bacteria can secrete virulence factors (Price et al., 2021). One example of a secreted virulence factor is the secretion of a proteolytic enteroxin, B. fragilis toxin (BFT). This toxin, secreted by Bacteroides fragilis, can cause diarrhea and intestinal inflammation through its activation of NF-kappa B pathways by triggering cleavage of E cadherin in host colonic epithelial cells (Valguarnera E, Wardenburg J B. Good gone bad: One toxin away from disease for Bacteroides fragilis. J Mol Biol. 2020; 432(4): 765-785).

These interactions between gut microbiota and the host have been shown to be especially important in the first few years of a child's life, when communication between immune cells and intestinal microbiota trains the immune system and shapes how cwCF respond to future infections (Kristensen et al., 2020; Hoen et al., 2015; Constantinides et al., 2019; Cahenzli J, Köller Y, Wyss M, Geuking M B, McCoy K D. Intestinal microbial diversity during early-life colonization shapes long-term IgE levels. Cell Host Microbe. 2013; 14(5):559-570; A1 Nabhani Z, Dulauroy S, Marques R, et al. A weaning reaction to microbiota is required for resistance to immunopathologies in the adult. Immunity. 2019; 50(5): 1276-1288.e5; Depner M, Taft D H, Kirjavainen P V., et al. Maturation of the gut microbiome during the first year of life contributes to the protective farm effect on childhood asthma. Nat Med. 2020; 26(11):1766-1775). Individuals with chronic lung diseases, such as asthma, emphysema, and CF have benefited from gut microbiome manipulation: it has previously been shown in both mouse models and clinical trials that manipulating the gut microbiome, whether by probiotics, prebiotics, or fecal microbial transplants, lung disease progression was attenuated (Jang Y O, Lee S H, Choi J J, et al. Fecal microbial transplantation and a high fiber diet attenuates emphysema development by suppressing inflammation and apoptosis. Exp Mol Med. 2020; 52(7):1128-1139; Neri L D C L, Taminato M, Da Silva Filho L V R F. Systematic review of probiotics for cystic fibrosis patients: Moving forward. J Pediatr Gastroenterol Nutr. 2019; 68(3): 394-399; van Dorst J M, Tam R Y, Ooi C Y. What do we know about the microbiome in cystic fibrosis? Is there a role for probiotics and prebiotics? Nutrients. 2022; 14(3): 1-27; Wen L, Shi L, Kong X-L, et al. Gut microbiota protected against Pseudomonas aeruginosa pneumonia via restoring Treg/Th17 balance and metabolism. Front Cell Infect Microbiol. 2022; 12(June):1-15).

Machine learning is increasingly being used with clinical data to assist disease diagnosis (Shah P, Kendall F, Khozin S, et al. Artificial intelligence and machine learning in clinical development: a translational perspective. NPJ Digit Med. 2019; 2(1)). In many cases, knowledge gained from machine learning can both assist physicians in making a diagnosis and help with design of patient-specific treatments. For example, such predictive approaches have been used to design treatment based on early signs of diabetes and Covid-19 (Shah et al., 2019; Anahtar M N, Yang J H, Kanjilal S. Applications of machine learning to the problem of antimicrobial resistance: an emerging model for translational research. J Clin Microbiol. 2021; 59(7):e0126020; AlJame M, Imtiaz A, Ahmad I, Mohammed A. Deep forest model for diagnosing COVID-19 from routine blood tests. Sci Rep. 2021; 11(1):1-12).

There remains a need for ways to identify patients with cystic fibrosis who have a high risk for more negative clinical outcomes, and to provide a therapeutic intervention to such patients to ameliorate, alleviate, reverse, or eliminate such clinical outcomes.

### SUMMARY

As an aspect of the present invention, a method is provided for reducing frequency and/or number of upper respiratory infections in a patient with cystic fibrosis. The method comprises identifying a patient with cystic fibrosis (CF) as having a high risk for upper respiratory infections (URIs) based on the patient's stool microbiota and providing a therapeutic intervention to the patient. The patient can be identified as having a high risk for frequent URIs or a high risk for numerous URIs, for example more than one URI per year. The high risk can determined based upon a relative abundance of one or more genera of stool microbiota, such as Faecalibacterium, Butyricoccus, or Bacteroides.

As another aspect, a method is provided for reducing systemic inflammation in a patient with cystic fibrosis based on the patient's stool microbiota. The method comprises identifying the patient as having systemic inflammation based on the patient's stool microbiota, and providing a therapeutic intervention to the patient. The patient can be identified as having systemic inflammation based upon a relative abundance of one or more genera of stool microbiota, such as Lactococcus, Kluyvera, or Klebsiella.

As yet another aspect, a computer-implemented system is provided for identifying a patient with cystic fibrosis as having a high risk for upper respiratory infections based on the patient's stool microbiota. The computer-implemented system comprises a machine learning model trained with data comprising (a) risk classifications for subjects with cystic fibrosis, and (b) stool microbiota information for each of the subjects. The subjects are classified as having (i) a high risk of upper respiratory infections (URIs), or (ii) a low risk of URIs and/or a medium risk of URIs. The trained machine learning model is configured to analyze a patient's stool microbiota information as input values, and to provide the patient's risk classification as an output value. In some embodiments, the subject is classified as high risk when the subject has a URI frequency of more than 1 per year, or when the subject has 2 or more URIs between 0 days to 2 years old. The patient's risk classification can be determined based upon a relative abundance of said one or more genera of stool microbiota, such as Faecalibacterium, Butyricoccus, or Bacteroides. The trained machine learning model can be configured to receive a patient's relative abundance of genera or amplicon sequence variants (ASV) for the stool microbiota as input values.

As another aspect of the present invention, a computer-implemented system is provided for identifying a patient with cystic fibrosis as having systemic inflammation based on the patient's stool microbiota. The computer-implemented system comprises a machine learning model trained with data comprising (a) risk classifications for subjects with cystic fibrosis, and (b) stool microbiota information for each of the subjects. The subjects are classified as having (i) high systemic inflammation, or (ii) medium systemic inflammation and/or low systemic inflammation. The trained machine learning model is configured to analyze a patient's stool microbiota information as input values, and to provide the patient's systemic inflammation as an output value. In some embodiments, the subjects are classified based on neutrophil-to-lymphocyte ratio (NLR) as a biomarker. The patient's risk classification can be determined based upon a relative abundance of said one or more genera of stool microbiota, such as Lactococcus, Kluyvera, or Klebsiella. The trained machine learning model can be configured to receive a patient's relative abundance of genera or ASVs for the stool microbiota as input values.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 shows Random Forest Model for Predicting Age from Stool Microbiota. (A) Normalized relative abundance of the top 10 genera (˜64% of total counts) in the stool samples collected from cwCF over the first four years of life. (B) Illustration of the pipeline for supervised machine learning through classification. Samples are labeled with their age and the normalized relative abundance of stool microbiota to train a random forest model to generate a predictive model for age. (C) Confusion matrix heatmap for the age prediction model. Overall OOB estimate of error rate for predicting age from stool microbiota is 45%. OOB is also shown for each indicated age. (D) Impact Factor summary plot for the top 5 stool microbiota important for classifying samples in the age model. Turicibacter, Bifidobacterium, and Lachnoclostridium are the top genera important for accurately predicting age of infant at which stool sample was collected.

FIG. 2 shows Correlating Microbes and Total URI. Correlation between total URI and log₁₀ average relative abundance of (A) Turicibacter, (B) Bifidobacterium, and (C) Lachnoclostridium in the first two years of life. Each dot represents a single patient. Turicibacter shows significant positive correlation with Total URI when Turicibacter is present, but Bifidobacterium and Lachnoclostridium do not. Spearman correlation performed with R and the p value is indicated.

FIG. 3 shows Association of Stool Microbiota with URI. (A) Confusion matrix heatmap for the URIfreq prediction model. The overall OOB estimate of error rate for predicting age from stool microbiota is 40%. Error rate for predicting High URIfreq is 16%. (B) The Impact Factor summary plot for the top 5 stool microbiota important for classifying samples in the age model. Faecalibacterium, Butyricoccus, and Bacteroides are the top genera important for accurately predicting URIfreq classification of cwCF. (C) Normalized relative abundance of top 10 genera (˜64% of total counts) in the stool samples collected from infants with CF for each URIfreq classification group. (D) PCoA depicting beta diversity distances between URIfreq groups. Differences in community structure assessed by Bray-Curtis dissimilarity was revealed by PERMANOVA (adonis test) as a function of URIfreq group (P=0.001).

FIG. 4 shows Association of Stool Microbiota with NLR. (A) Confusion matrix heatmap for the NLR prediction model. The overall OOB estimate of error rate for predicting NLR from stool microbiota is 64% but error rate for predicting High NLR is 27%. (B) Impact Factor summary plot for the top 5 genera important for classifying samples in the NLR model. Lactococcus is the top genus important for accurately predicting NLR classification group in pwCF. (C) Normalized relative abundances of top 10 genera (˜64% of total counts) in the stool samples collected from infants with CF for each NLR classification group. (D) PCoA depicting beta diversity distances between NLR groups. Statistically different community structure assessed by Bray-Curtis dissimilarity was revealed by PERMANOVA (adonis test) as a function of NLR groups. Composition of NLR groups is systematically different (P==0.008). However, factors other than NLR seem to drive most of the differences in composition because NLR explains little of the variability in composition (R2=0.085) as determined by PERMANOVA.

FIG. 5 shows Age Model Impact Factors and Node Purity. (A) Mean Decrease Accuracy summary plot for the top 10 stool microbial taxa important for classifying samples in the age model. B) Mean Decrease Gini summary plot for the top 10 stool microbiota with highest node purity.

FIG. 6 shows URIfreq Model Impact Factors and Node Purity. (A) Mean Decrease Accuracy summary plot for the top 10 stool microbial taxa important for classifying samples in the URIfreq model. B) Mean Decrease Gini summary plot for the top 10 stool microbial taxa with highest node purity in the URIfreq model. (C) Mean Decrease Accuracy summary plot for the top 10 stool microbial taxa important for classifying samples in the URIfreq model trained on ASVs combined at a phylum level.

FIG. 7 shows Association of Microbial Composition and URI. (A) Chao-1 alpha diversity of stool microbiota in URIfreq groups. There is a significant increase in Chao-1 alpha diversity between the Low and High groups of URIfreq. (B) Shannon-alpha diversity of stool microbiota in URIfreq groups. Statistically determined by Wilcoxon rank sum with FDR correction for multiple testing. (C) PCoA plot depicting beta diversity distances between URIfreq groups at each year of life. Composition of URIfreq groups is systematically different when the interaction of age is accounted for (P==0.009), as determined by PERMANOVA.

FIG. 8 shows NLR Model Impact Factors and Node Purity. (A) Mean Decrease Accuracy summary plot for the top 10 stool microbial taxa important for classifying samples in the NLR model. B) Mean Decrease Gini summary plot of the top 10 microbial taxa with highest node purity in the NLR model.

FIG. 9 shows Association of Microbial Composition and NLR. (A) Chao-1 alpha diversity of stool microbiota in NLR groups. (B) Shannon-alpha diversity of stool microbiota in NLR groups. Statistical significance determined by Wilcoxon rank sum with FDR correction for multiple testing.

### DETAILED DESCRIPTION

The present disclosure provides, in part, the use of supervised machine learning to train a random forest model on the distinct microbial composition of cwCF to predict: age (as a validation of the method), frequency of upper respiratory infection (URIfreq), and neutrophil to lymphocyte ratio (NLR), a clinical marker for systemic inflammation that negatively correlates with lung function. The out of bag error, a measure of model accuracy, is lower when predicting age for cwCF compared to children without CF, consistent with previous data. High URIfreq is predicted with only 16% error and high NLR with 27% error. This machine learning pipeline may allow physicians and microbiome researchers to use the stool microbiota of cwCF as a tool for identifying individuals with the more negative airway clinical outcomes from this population, and potentially allow for early intervention.

Children with CF (cwCF) often experience chronic respiratory infections, leading to progressive, irreversible lung function decline and significant morbidity and premature mortality. Modulator therapy has revolutionized the treatment of eligible adults with CF. cwCF as young as age 6 are now eligible to receive modulator therapy, although by early childhood many children have already experienced pulmonary exacerbations. The present disclosure shows that for cwCF, stool microbiota composition is associated with higher upper respiratory infection frequency and increased systemic inflammation. The present findings may aid in developing diagnostic tools that can allow physicians further understanding of which intestinal microbiota profiles are associated with health outcomes and to identify targets for preventative treatment for cwCF.

The present disclosure used longitudinal stool and clinical data collections to train a model that can predict respiratory outcomes for children with CF from stool samples. Overall, the relative abundance of stool microbiota at the genus level in children with CF weakly predicts age, consistent with studies showing intestinal microbiome dysbiosis and altered age of maturation in this population (Price et al., 2021; De Lisle et al., 2013; Hoffman et al., 2014; Kristensen et al., 2020; Hayden et al., 2020; Meeker et al., 2020; Hoen et al., 2015; Neri et al., 2019; van Dorst et al., 2022; Price C E, Valls R A, Ramsey A R, et al. Intestinal Bacteroides modulates systemic inflammation and the microbial ecology in a mouse model of CF: Evidence for propionate and other short chain fatty acids reducing systemic inflammatory cytokines. bioRxiv. January 2022:2022.01.05.475125; Bazett M, Bergeron M E, Haston C K. Streptomycin treatment alters the intestinal microbiome, pulmonary T cell profile and airway hyperresponsiveness in a cystic fibrosis mouse model. Sci Rep. 2016; 6(August 2015):1-13). Specifically, weak predictive power in cwCF compared to the general population suggests that the CF infant microbiome may mature at different rates than in infants without CF. Nonetheless, the genus of bacteria most important for predicting age in cwCF was Turicibacter.

In a previous report (Hayden et al., 2020), Blautia was identified as a genus important for model accuracy for age prediction. In our analysis, Blautia also was identified as an important genus for predicting age prior to sub-sampling to balance the numbers of cwCF in each of the age groups. Upon subsampling, so that an even 21 samples per age group were used to train the model, Turicibacter was the top genus important for predicting age in infants with CF. However, the experiment of Example 1 did not identify Blautia as a top genus for predicting age after subsampling. The Hayden Cohort (Hayden et al., 2020) consisted of cwCF ages 3 to 12 months. From this, it is surmised that Blautia may be important for predicting age in children in the first year of life but less so post 12 months of age. The genera Blautia and Turicibacter have also been identified as top contributors for predicting age from stool microbiota in children with asthma in the first year of life (Depner et al., 2020).

In addition to its importance for predicting age, in the experiment reported in Example 3, Turicibacter was also found to have a positive correlation with total URI. Turicibacter has previously been shown to correlate with a decrease in butyrate in the CF gut (van Dorst et al., 2022), is considered a top contributor of acetate (Depner et al., 2020), and has been shown to positively correlate with inflammatory markers in asthmatic individuals (Li L, Fang Z, Liu X, et al. Lactobacillus reuteri attenuated allergic inflammation induced by HDM in the mouse and modulated gut microbes. PLOS One. 2020; 15(4): 1-14). Thus, for the subset of individuals with Turicibacter (in only a subset of cwCF was this genus detected), this microbe could serve as a biomarker to calibrate “microbiota age” and perhaps help predict the frequency of negative respiratory outcomes.

Of clinical relevance, applying a random forest model to the stool microbiome of infants with CF could also predict cwCF in the “high” URIfreq group with relatively low error. The model's predictive power for the high URIfreq group supports the idea that this pipeline can be used to identify children who are at a higher risk for upper respiratory infection and therefore pulmonary exacerbations that drive rapid lung function decline. Significant differences were observed in richness of the communities associated with the “high” URIfreq group, but no difference in evenness of beta diversity. These data likely indicate a complex relationship between intestinal gut microbiota composition and specific airway clinical outcomes.

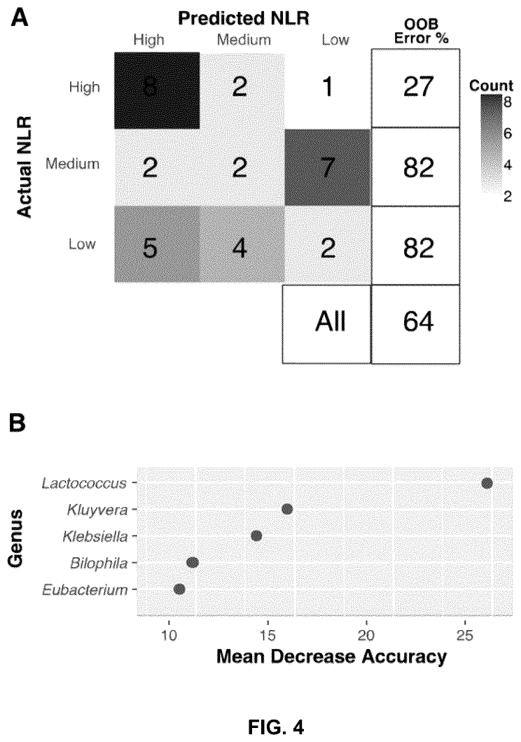

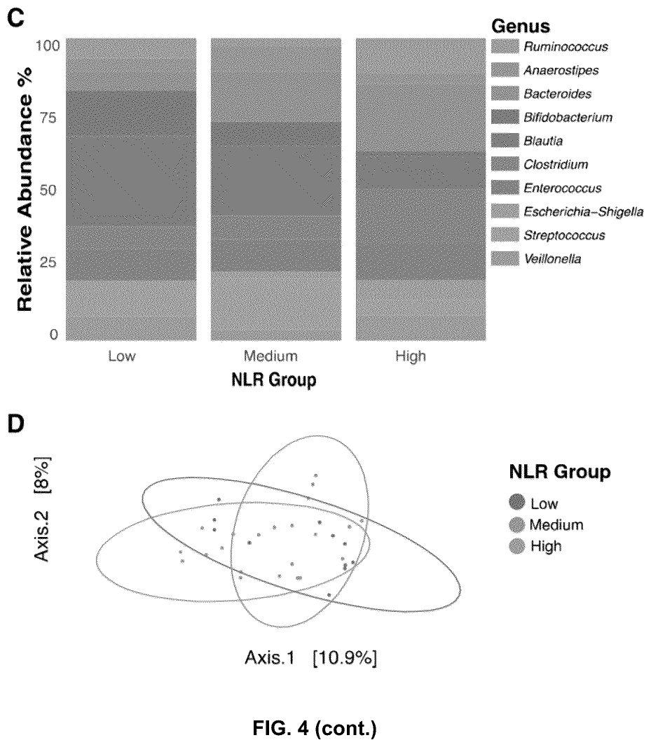

Because previous literature has shown that the microbes in the gut can alter systemic inflammatory profiles (Kristensen et al., 2020; Yoon et al., 2018; Stefan et al., 2020; Constantinides et al., 2019; Cahenzli et al., 2013; Depner et al., 2020; Price et al., 2022; Bazett et al., 2015; Li et al., 2020), the machine learning predictive pipeline was applied to markers of systemic inflammation, as described in Example 4. Again, the model was able to correlate stool microbial composition with the High NLR group (i.e., the highest systemic inflammation) with relatively low error. It has been shown that Bacteroides is depleted in the gut of infants and cwCF (Price et al., 2021; De Lisle et al., 2013; Kristensen et al., 2020; van Dorst et al., 2022; Price et al., 2022). While the levels of Bacteroides are low compared to healthy controls for all our samples (see Antosca et al., 2019), it was unexpectedly observed that the High NLR group had the highest relative abundance of Bacteroides (See Example 4 below, and panel C in FIG. 4). This somewhat surprising finding indicates that other microbes might be driving this clinical outcome, a finding consistent with the Impact Factor analysis shown in panel B in FIG. 4. Additionally, previous studies have shown a positive relationship between Bacteroides eggerthii and NLR in adults (see Anahtar et al., 2021), indicating that if this species of Bacteroides is present, it might be driving the observed outcome. Furthermore, the High NLR group also shows an increase of 200% in Clostridium relative abundance, a genus known to be elevated in CF mice (see Bazett et al., 2015). Together, these data could be used to generate hypotheses to explore the mechanisms whereby these microbial communities may be driving the observed clinical outcomes.

One priority of CF research is to decrease the high burden of medical procedures that cwCF experience. The present disclosure provides proof of concept that stool samples, collected in diapers by parents or physicians in regular clinical visits, could identify children with the highest likelihood of negative respiratory outcomes. Analyzing stool samples could replace the use of uncomfortable oropharyngeal sampling or respiratory lavage samples for diagnostics. By training a predictive model on stool microbiota composition, the aim was to predict likelihood of cwCF having frequent upper respiratory infections and/or showing high levels of systemic inflammation. Applying such approaches to CF would build on previous use of such tools in the context of other diseases (Shah et al., 2019; Anahtar et al., 2021; AlJame et al., 2021; Ravaut M, Harish V, Sadeghi H, et al. Development and validation of a machine learning model using administrative health data to predict onset of Type 2 diabetes. JAMA Netw Open. 2021; 4(5):1-15). For example, machine learning applied to predictive modeling was able to prevent 30% of inappropriate antibiotic therapies in a randomized control trial (Anahtar et al., 2021). Thus, the feasibility of using stool samples to predict clinical outcomes has promise in CF.

### I. COMPUTER-IMPLEMENTED SYSTEMS

In some embodiments, the present disclosure provides computer-implemented systems for identifying a patient with cystic fibrosis as having a high risk for upper respiratory infections based on the patient's stool microbiota. The computer-implemented system comprises a machine learning model trained with data comprising (a) risk classifications for subjects with cystic fibrosis (CF), and (b) stool microbiota information for each of the subjects. The subjects are classified as having (i) a high risk of upper respiratory infections (URIs), or (ii) a low risk of URIs and/or a medium risk of URIs. The trained machine learning model is configured to analyze a patient's stool microbiota information as input values, and to provide the patient's risk classification as an output value. In some embodiments, the subject is classified as high risk when the subject has a URI frequency of more than 1 per year, or when the subject has 2 or more URIs between 0 days to 2 years old.

In some embodiments, the present disclosure provides computer-implemented systems for identifying a patient with cystic fibrosis as having systemic inflammation based on the patient's stool microbiota. The computer-implemented system comprises a machine learning model trained with data comprising (a) risk classifications for subjects with cystic fibrosis, and (b) stool microbiota information for each of the subjects. The subjects are classified as having (i) high systemic inflammation, or (ii) medium systemic inflammation and/or low systemic inflammation. The trained machine learning model is configured to analyze a patient's stool microbiota information as input values, and to provide the patient's systemic inflammation as an output value. In some embodiments, the subjects are classified based on neutrophil-to-lymphocyte ratio (NLR) as a biomarker.

The machine learning models can be generated using training data from a plurality of subjects with known classifications of URI risk or systemic inflammation, and their associated stool microbiota information. In the present disclosure, “subject” usually refers to an individual whose data is used for the training of a machine learning model, while “patient” usually refers to an individual to be identified using the present methods and systems as having a high risk of upper respiratory infection or as having systemic inflammation. Machine learning is rooted in computer technology and cannot be implemented in the absence of computing technology or performed in the human mind. While machine learning systems utilize various types of statistical analyses, machine learning is distinguished from mathematical formulas and concepts by virtue of the ability to learn and being rooted in computer technology.

In some embodiments, the machine learning model comprises a linear classifier (such as logistic regression or naïve Bayes classifier), a decision tree (such as a random forest model), or a neural network. In some embodiments, the machine learning model comprises a classifier trained and constructed according to one or more of: random forest, linear discriminant analysis (LDA), partial least squares (PLS), k-nearest neighbor (KNN), support vector machine (SVM) with radial basis function kernel (SVMRadial), SVM with linear basis function kernel (SVMLinear), SVM with polynomial basis function kernel (SVMPoly), decision trees, multilayer perceptron, mixture of experts, sparse factor analysis, hierarchical decomposition, and combinations of linear algebra routines and statistics. In some embodiments, the machine learning model is a random forest model.

In some embodiments of the computer-implemented systems for identifying a patient with cystic fibrosis as having a high risk for upper respiratory infections, the patient's risk classification can be determined based upon a relative abundance of one or more genera of stool microbiota, such as Faecalibacterium, Butyricoccus, or Bacteroides. In some embodiments, all three of those genera are used to determine the patient's risk classification. In some embodiments, the patient's risk classification is determined based upon relative abundance of genera of stool microbiota, wherein said genera include at least 2, 3, 4, 5, 6, 7, 8, 9 or all of the following genera: Faecalibacterium, Butyricicoccus, Bacteroides, Lachnospiraceae, Dielma, Sutterella, Faecalicoccus, Dorea, Staphylococcus, and Prevotella, In some embodiments, all three of those genera are used to determine the patient's risk classification, and/or genera other than those three are used to determine the patient's risk classification.

In some embodiments of the computer-implemented systems for identifying a patient with cystic fibrosis as having systemic inflammation, the patient's risk classification can be determined based upon a relative abundance of one or more genera of stool microbiota, such as Lactococcus, Kluyvera, or Klebsiella. In some embodiments, all three of those genera are used to determine the patient's risk classification, and/or genera other than those three are used to determine the patient's risk classification. In some embodiments, the patient's risk classification is determined based upon relative abundance of genera of stool microbiota, wherein said genera include at least 2, 3, 4, 5, 6, 7, 8, 9 or all of the following genera: Lactococcus, Kluyvera, Klebsiella, Bilophila, Eubacterium, Corynebacterium, Flavonifractor, Bifidobacterium, Terrisporobacter, and Anaerococcus,

Stool microbiota information for individuals (subjects and/or patients) can be obtained in any manner. In some embodiments, the stool microbiota information is obtained experimentally and/or personally, either by the individual's healthcare provider after collecting a stool sample from the individual, or from a laboratory which receives and analyzes a sample of an individual's stool and provides the stool microbiota information to the individual, to a healthcare provider, or to an appropriate collector of such information. In some embodiments, stool microbiota information is obtained by collecting DNA from an individual's stool sample and performing nucleotide sequencing of the collecting DNA. For instance, stool microbiota information can be obtained by metagenomic sequencing of bacterial DNA from the sample to generate sequencing reads; and mapping the reads map to one or more genera; and determining a relative amount of different sequences in the sample that correspond to a sequence from the one or more genera.

In some embodiments, stool microbiota information and risk classifications for subjects to be used in training the machine learning model is obtained from data sources provided by appropriate collectors of such data. For instance, information for classifying individuals as having a risk of URI or systemic inflammation may be collected from many different sources, such as electronic medical records (EMR), electronic health records (EHR), insurance claims data, and academic studies. Data sources may be any suitable sources of data, for example, databases, web data servers, and proprietary databases such as those associated with specific type of data including, but not limited to the CF Foundation Patient Registry, the UK Cystic Fibrosis Registry, GenomicsDB, PubMed, Amazon EMR, the Biological Specimen and Data Repository Information Coordinating Center, the Biomedical Translational Research Information System, and others.

Stool microbiota information can include taxonomic information related to operational taxonomic units (OTUs), amplicon sequence variants (ASVs), diversity metrics (e.g., alpha-diversity metrics, beta-diversity metrics, gamma-diversity metrics, etc.), and/or quantifications of various taxonomic units (e.g., relative quantifications, absolute quantifications). In some embodiments, the stool microbiota information includes data indicating presence, absence or abundance (relative or absolute) of one or more genera of stool microbiota. The stool microbiota information can determined by techniques such as quantitative polymerase chain reaction (qPCR), sequencing of bacterial 16S rRNA gene, and shotgun metagenome sequencing. The specific genetic sequence used to define an OTU or ASV may be the 16S rRNA sequence or a portion of the 16S rRNA sequence or it may be a functionally conserved gene found broadly across the bacterial domain. For example, relative abundance of the genera in an individual's stool microbiota can be based on 16S rRNA sequencing data, such as operational taxonomic units and amplicon sequence variants defined by 16S rRNA sequences. In some embodiments, the 16S rRNA sequencing or whole genome/metagenomics sequencing is used to determine the relative abundance of genera of the stool microbiota in the sample. Additional details about techniques for characterizing microbiota through sequencing can be found in Apte et al. US Pat. App. Publication No. 20190050534 A1 and Raman et al., US Pat. App. Publication No. 20200411144 A1.

In some embodiments, the trained machine learning models of the present disclosure are configured to receive microbiota sequence data indicating relative abundance of genera or of genus-identifying regions in the patient's stool microbiota as input values. In some embodiments, the microbiota sequence data comprises amplicon sequence variant (ASV) region sequence data.

The present invention may be implemented in various ways, including as novel computer-implemented systems, or by using computer-implemented system to perform the methods described below: In some embodiments, the computer-implemented system comprises a processor and a memory medium which stores a plurality of instructions which, when executed by the processor, cause the processor to apply the trained machine learning model to the patient's stool microbiota information and to provide the patient's category of systemic inflammation or risk of URI.

Such computer-implemented systems may include one or more software components including, for example, software objects, methods, data structures, or the like. A software component may be coded in any of a variety of programming languages, such as a lower-level programming language or a higher-level programming language. Examples of lower-level programming languages include an assembly language associated with a particular hardware architecture and/or operating system platform. A software component comprising assembly language instructions may require conversion into executable machine code by an assembler prior to execution by the hardware architecture and/or platform. Examples of higher-level programming languages include those that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In some embodiments, a software component comprising instructions in one of the foregoing programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

The present computer-implemented systems may comprise a non-transitory computer-readable memory medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable memory media include all computer-readable media (including volatile and non-volatile media).

### II. METHODS OF REDUCING URIS OR SYSTEM INFLAMMATION IN A CYSTIC FIBROSIS PATIENT

In some embodiments, the present disclosure provides methods for reducing frequency and/or number of upper respiratory infections in a patient with cystic fibrosis. The method comprises identifying a patient with cystic fibrosis (CF) as having a high risk for upper respiratory infections (URIs) based on the patient's stool microbiota, and providing a therapeutic intervention to the patient. The patient can be identified as having a high risk for frequent URIs or a high risk for numerous URIs, for example more than one URI per year. The high risk can determined based upon a relative abundance of one or more genera of stool microbiota, such as Faecalibacterium, Butyricoccus, or Bacteroides.

In some embodiments, the present disclosure provides methods for reducing systemic inflammation in a patient with cystic fibrosis (CF). The method comprises identifying the patient as having systemic inflammation based on the patient's stool microbiota, and providing a therapeutic intervention to the patient. The patient can be identified as having systemic inflammation based upon a relative abundance of one or more genera of stool microbiota, such as Lactococcus, Kluyvera, or Klebsiella.

The patient's stool microbiota information can be obtained as described above, such as by genetic sequencing. The present methods can also comprise collecting a stool sample from the patient; extracting microbiota nucleic acids from the stool sample; sequencing a genus-identifying region of the microbiota nucleic acids to provide microbiota identifying information; and determining the relative abundance of the one or more genera or the genus-identifying regions of stool microbiota based on the microbiota sequence data. In some embodiments, the genus-identifying region is an amplicon sequence variant (ASV) region, such as an ASV region comprises a V4-V5 region of 16S rRNA gene.

In some embodiments, the patient is 1 week old to 6 months old, alternatively 1 week old to 12 months old, alternatively 1 week old to three years old, alternatively 1 week old to four years old.

In some embodiments, the method comprises obtaining a stool sample from a patient. In some embodiments, the stool sample is obtained from a patient age 2 or less, age 18 months or less, age 12 months or less, age 6 months or less, or age 4 months of less.

### III. THERAPEUTIC INTERVENTIONS BASED ON PATIENT CLASSIFICATIONS

In some embodiments, identification of a patient's risk of upper respiratory infection or systemic inflammation helps physicians or other healthcare providers to determine how aggressively to treat a patient's cystic fibrosis and which therapeutic interventions may be appropriate. A patient who has higher risk of URI and/or systemic inflammation is more susceptible to disease-associated difficulties and should be treated more aggressively.

The patient classification approaches as outlined above may be followed, when appropriate, with therapeutic interventions as described herein or as determined by a healthcare provider for the patient. Specifically, patient classification allows a healthcare provider to determine the need for therapeutic intervention (including preventive therapies) for cystic fibrosis or cystic fibrosis-associated clinical outcomes. Conventional treatments, therapies, or lifestyle changes may be administered or prescribed to a subject in need thereof.

In some embodiments, the therapeutic intervention reduces a risk of advanced stage lung disease for the patient or a risk of the patient requiring a lung transplant. In some embodiments, the therapeutic intervention comprises altering an intestinal microbiome of the patient. In some embodiments, the therapeutic intervention reduces a risk of upper respiratory infection selected from the group consisting of Staphylococcus aureus infection, Pseudomonas aeruginosa infection, Stenotrophomonas spp. infection, Streptococcus spp. infection, Haemophilus influenzae infection, nontuberculous Mycobacterium infection, Burkholderia cepacia complex infection, viral infection, fungal infection and a co-infection with multiple pathogens.

In some embodiments, the present methods comprise providing a therapeutic intervention to an individual. By way of example, the therapeutic intervention can include (i) administering bacterial compositions, pancreatic enzyme replacement, probiotics, prebiotics, antibiotics, anti-inflammatory medications, mucus-thinning drugs, cystic fibrosis transmembrane conductance regulator (CFTR) function-improving medications, inhaled medications or bronchodilators; and/or (ii) providing physical therapy including postural drainage and chest physical therapy, pulmonary rehabilitation to the subject, or fecal microbiota transplantation (FMT) to a patient.

### A. Bacterial Compositions

In some embodiments, bacterial compositions, as described herein and in O'Toole et al., U.S. Pat. No. 11,351,208 B2, may be administered as the therapeutic intervention. The bacterial compositions can include one or more bacteria of the genera Bacteroides or one or more bacteria of the genera Bifidobacterium or one or more bacteria of the genera Faecalibacterium, or one or more bacteria of the family Clostridiales or one or more bacteria of the genera Roseburia or one or more bacteria of the family Ruminococcaceae or one or more bacteria of the genera Eubacterium or one or more bacteria of the genera Streptococcus or one or more bacteria of the genera Ruminococcus.

In some embodiments, bacterial compositions as described herein include two or more bacteria of the genera/family Bacteroides, Bifidobacterium, Faecalibacterium, Clostridiales, Roseburia, Ruminococcaceae, Eubacterium, Streptococcus and Ruminococcus.

In some embodiments, bacterial compositions as described herein include three or more bacteria of the genera/family Bacteroides, Bifidobacterium, Faecalibacterium, Clostridiales, Roseburia, Ruminococcaceae, Eubacterium, Streptococcus and Ruminococcus.

In some embodiments, bacterial compositions as described herein include four or more bacteria of the genera/family Bacteroides, Bifidobacterium, Faecalibacterium, Clostridiales, Roseburia, Ruminococcaceae, Eubacterium, Streptococcus and Ruminococcus.

In some embodiments, bacterial compositions as described herein include five or more bacteria of the genera/family Bacteroides, Bifidobacterium, Faecalibacterium, Clostridiales, Roseburia, Ruminococcaceae, Eubacterium, Streptococcus and Ruminococcus.

In some embodiments, bacterial compositions as described herein include six or more, seven or more, eight or more, or all nine of the genera/family Bacteroides, Bifidobacterium, Faecalibacterium, Clostridiales, Roseburia, Ruminococcaceae, Eubacterium, Streptococcus and Ruminococcus.

In any of the embodiments described herein, the bacterial composition can comprise any combination of two, three, four, five six, seven, eight or nine bacteria of the genera/family Bacteroides, Bifidobacterium, Faecalibacterium, Clostridiales, Roseburia, Ruminococcaceae, Eubacteriums, Streptococcus and Ruminococcus.

In some embodiments, bacterial compositions as described herein include both Bacteroides and Bifidobacterium. In some embodiments, bacteria of the genera Bifidobacterium include Bifidobacterium theta.

In some embodiments, bacterial compositions as described herein include one or more bacteria of the genera Streptococcus or one or more bacteria of the family Ruminococcus.

In some embodiments, bacterial compositions as described herein include one or more bacteria of the genera Streptococcus.

In some embodiments, bacteria of the genera Streptococcus include one or more of S. bovis, S. gallolyticus, S. macedonicus, S. alactolyticus, S. vestibularis, S. salivarius, S. mitis, S. parasanguinis, S. lutetiensis, S. equinus, S. infantarius, or operational taxonomic unit (OTU) encompassing said species.

In some embodiments, bacterial compositions as described above do not comprise bacteria of the family Clostridiales.

In some embodiments, a bacterial composition as described herein include bacteria as described herein, present in treated fecal material from a healthy donor or individual that does not have CF or a patient diagnosed with or treated for cystic fibrosis. Such bacterial compositions may be “directly isolated” and not resulting from any culturing or other process that results in or is intended to result in replication of the population after obtaining the fecal material. In some embodiments, bacteria as described herein include bacterial spores. In some embodiments, the bacterial compositions may be obtained from single bacterium or mixed bacteria having been grown in culture.

In some embodiments, a bacterial composition as described herein includes human bacterial strains. In alternative embodiments, a bacterial composition as described herein includes bacterial strains not generally found in humans.

In some embodiments, a bacterial composition as described herein includes bacteria capable of colonizing the gastrointestinal tract of a subject receiving the bacterial composition.

In some embodiments, a bacterial composition as described herein includes live bacteria.

In some embodiments, a bacterial composition as described herein includes substantially pure bacteria of the genera/family Bacteroides, Bifidobacterium, Faecalibacterium, Clostridiales, Roseburia, Ruminococcaceae, Eubacterium, Streptococcus and/or Ruminococcus. By “substantially pure” or “isolated” is meant bacteria of the genera/family Bacteroides, Bifidobacterium, Faecalibacterium, Clostridiales, Roseburia, Ruminococcaceae, Eubacterium, Streptococcus and/or Ruminococcus that are separated from the components that naturally accompany it, in for example, fecal matter or in the gastrointestinal tract. Typically, a bacterial composition as described herein is substantially pure when it is at least 50%, 60%, 70%, 75%, 80%, or 85%, or over 90%, 95%, or 99% by weight, of the total material in a sample. A substantially pure bacterial composition, as described herein, can be obtained, for example, by extraction from a natural source, such as fecal material, gastrointestinal tract, or other bodily fluid or material, or from bacterial cultures, for example, cultures of any of the bacteria described herein, such as Bacteroides, Bifidobacterium, Faecalibacterium, Clostridiales, Roseburia, Ruminococcaceae, Eubacterium, Streptococcus and/or Ruminococcus. By “substantially pure” or “isolated” the application can be referring to the component bacteria that make up the culture (adding, for example, Bacteroides that is 95% pure) or it can refer to the entire bacterial composition (the bacterial composition is 95% pure, meaning 95% comes from the component bacteria when added together, such as 50% Bacteroides, 30% Bifidobacterium, and 15% Faecalibacterium, and only 5% of components that natural accompany the bacteria such as fecal matter).

In some embodiments, a substantially pure bacterial composition, as described herein, is obtained, for example, by extraction from a natural source, such as fecal material, gastrointestinal tract, or other bodily fluid or material, from a healthy individual, an individual who does not have CF, or an individual who is being treated for or has been diagnosed with CF.

### B. Microbiome Treatment

Bacterial compositions, as described herein and in O'Toole et al., U.S. Pat. No. 11,351,208, can be used as the therapeutic intervention of the present methods to alter the intestinal microbiota or to treat or protect against disease progression in cystic fibrosis, in particular, upper respiratory infection or systemic inflammation, in a subject in need thereof. Such other pathogenic microbial infections include, without limitation, infections with Staphylococcus aureus, Pseudomonas aeruginosa, Stenotrophomonas spp., Haemophilus influenzae, nontuberculous Mycobacterium (including but not limited to Mycobacteria abscessus or Mycobacteria avium), Burkholderia cepacia complex, viral or fungal infection, or co-infections with multiple pathogens. In some embodiments, treating or protecting against disease progression in cystic fibrosis results in the prevention of pulmonary exacerbation or P. aeruginosa or other pathogenic microbial infection in the subject. In some embodiments, a bacterial composition, as described herein, is used to alter intestinal microbiota or to treat or protect against other pathogenic microbial infection associated with pulmonary exacerbation, in a subject in need thereof.

In some embodiments, a bacterial composition, as described herein, may be a therapeutic (including prophylactic) composition.

In some embodiments, a bacterial composition may be a therapeutic (including prophylactic) composition including one or more bacteria of the genera/family Bacteroides, Bifidobacterium, Faecalibacterium, Clostridiales, Roseburia, Ruminococcaceae, Eubacterium, Streptococcus and/or Ruminococcus.

An “effective amount” of a bacterial composition, as used herein, includes an amount sufficient to colonize the gastrointestinal tract of a subject for a suitable period of time as determined, for example, by detecting the presence of one or more bacteria of the genera/family Bacteroides, Bifidobacterium, Faecalibacterium, Clostridiales, Roseburia, Ruminococcaceae, Eubacterium, Streptococcus and/or Ruminococcus, or their metabolic byproducts associated with clinical outcomes (including, without limitation, butyrate), in a sample, such as a stool sample, from the subject at specific periods after administration.

Conventional pharmaceutical or nutraceutical practice may be employed to provide suitable formulations or compositions to administer a bacterial composition, as described herein, to subjects suffering from or presymptomatic for cystic fibrosis, pulmonary exacerbation, P. aeruginosa infection, or any other CF-associated clinical outcomes. Any appropriate route of administration may be employed, for example, dermal, intranasal, inhalation aerosol, topical, gavage, rectal or oral administration.

The bacterial compositions can be in a variety of forms. These forms include, e.g., lyophilized, liquid, semi-solid and solid dosage forms, such as liquid solutions, dispersions or suspensions, tablets, pills, powders, liposomes, and suppositories. The preferred form depends, in part, on the intended mode of administration and application. Administration includes oral administration, nasogastric administration, rectal administration, and other forms of administration to the gastrointestinal tract.

Bacterial compositions, as described herein, can be formulated as a nutraceutical composition, such as medical foods, nutritional or dietary supplements, food products or beverage products, and include a nutraceutically acceptable carrier. As used herein, a “nutraceutically acceptable carrier” refers to, and includes, any and all solvents, dispersion media, coatings, antibacterial and antifungal agents, isotonic and absorption delaying agents, and the like that are physiologically compatible. The compositions can include a nutraceutically acceptable salt, e.g., an acid addition salt or a base addition salt. In some embodiments, the nutraceutically acceptable carrier is suitable for pediatric use.

Bacterial compositions, as described herein, can be formulated as a pharmaceutical composition and include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. In some embodiments, the pharmaceutically acceptable carrier is suitable for pediatric use.

### C. Prebiotics

In some embodiments, the therapeutic intervention comprises administering one or more prebiotics to the patient. Prebiotics can be administered in therapeutically effective amounts to promote the growth of one or more of the bacteria in the bacterial compositions described herein may be used. A prebiotic is usually a nondigestible carbohydrate or a sugar alcohol which is not degraded or absorbed in the digestive tract. The prebiotic is selected according to the bacterial compositions such that it supports the growth of the bacteria. Suitable prebiotics may include, e.g., oligosaccharides, particularly inulin, fructooligosaccharide (FOS), galactooligosaccharide (GOS), palatinoseoligosaccharide, soybean oligosaccharide, gentiooligosaccharide, xylooligomers, non-degradable starch, lactosaccharose, lactulose, lactitol, maltitol, polydextrose, pectin, or the like.

In some embodiments, such prebiotics include, without limitation, extracts of Gum arabic, leeks, asparagus, artichoke, chicory root, onion, garlic, kale, wheat bran, banana, oats, barley, various legumes, or the like. In some embodiments, prebiotics may include herbs. In some embodiments, such prebiotics may be commercially available prebiotic supplements.

### D. Antibiotics

In some embodiments, the therapeutic intervention comprises administering one or more antibiotics to the patient. Antibiotics may be administered in therapeutically effective amounts to a patient for treating cystic fibrosis or cystic fibrosis-associated clinical outcomes as described herein, particularly an upper respiratory infection. Such antibiotics may include, without limitation, penicillin (methicillin, oxacillin, naficillin, cabencillin, amoxicillin, clavulanic acid, cloxacillin, dicloxacillin, ticarcillin, piperacillin, mezlocillin, azlocillin, tazobactam); cephalosporins (cephalexin, cefdinir, cefprozil, ceflacor, cefuroxime, cefepime); sulfa antibiotics (sulfamethoxazole, trimethoprim); aminoglycosides (tobramycin, amikacin, gentamicin); erythromycin/sulfisoxazole; macrolides (erythromycin, clarithromycin, azithromycin); tetracyclines (tetracycline, doxycycline, minocycline, tigecycline); vancomycin; imipenem; meripenem; colistimethate); quinolones (ciprofloxacin, levofloxacin); aztreonam; linezolid. In some embodiments, one or more of Gentak, Cetraxal, Ciloxan, Cipro in D5W, Otiprio, Cipro XR (ciprofloxacin), Zithromax (azithromycin), AzaSite, Zmax, Zithromax TRI-PAK, Zithromax Z-Pak, Cayston, Azactam, Merrem, Fortaz, Tobi (tobramycin), Kitabis Pak, Bethkis, and Zosyn is used.

### E. Anti-Inflammatory Medications

In some embodiments, the therapeutic intervention comprises administering one or more anti-inflammatory medications to the patient. Anti-inflammatory medications can be administered in therapeutically effective amounts to lessen the inflammatory responses (e.g., swelling in the airways of the lungs) may be employed for the method for treating cystic fibrosis or cystic fibrosis-associated clinical outcomes. Such anti-inflammatory medications may include, without limitation, corticosteroid, ibuprofen or other non-steroidal anti-inflammatory medications, anti-inflammatory cytokines, antibody or antigen binding fragment thereof to pro-inflammatory cytokine, antioxidants, protease inhibitors, membrane stabilizers. Further, such anti-inflammatory medications may include, without limitation, corticosteroid (fluticasone (Xhance®) or prednisone (Deltasone®, Rayos®, Prednisone Intensol)), ibuprofen, Anti-ICAM-1, anti-IL-8, anti-IL-17, IL-10, Interferon-γ, p38 Mitogen-activated protein kinase inhibitors, al-Protease inhibitor, CXCR2 antagonist, cyclosporine-A, DHA, EPI-hNE4, glutathione, hydroxychloroquine, 1-Arginine, LTB4 receptor antagonist (BIIL 284 BS), methotrexate, monocyte/neutrophil elastase inhibitor, montelukast, N-Acetylcysteine, omega-3-fatty acids, secretory leukoprotease inhibitor, Simvastatin, thiazolidinediones/pioglitazone, vitamins C, E, and β-carotene, and vitamin D.

### F. Mucus-Thinning Drugs

In some embodiments, the therapeutic intervention comprises administering one or more mucus-thinning drugs to the patient. Mucus-thinning drugs can be administered in therapeutically effective amounts to improve airway function, thereby treating cystic fibrosis or cystic fibrosis-associated clinical outcomes. The mucus-thinning drugs include, without limitations, hypertonic saline, dornase alfa (Pulmozyme®), and acetylcysteine (Acetadote®, NAC®, and Cetylev®).

G. Medications that Improve Cystic Fibrosis Transmembrane Conductance Regulator (CFTR) Function

In some embodiments, the therapeutic intervention comprises administering one or more medications that improve cystic fibrosis transmembrane conductance regulator (CFTR) function, the defective protein that causes cystic fibrosis. Such medications can be administered in therapeutically effective amounts for treating cystic fibrosis or cystic fibrosis-associated clinical outcomes. In some embodiments, such medications include, without limitation, at least one of ivacaftor, lumacaftor/ivacaftor (ORKAMBI®), and tezacaftor/ivacaftor (SYMDEKO®), and elexacaftor/tezacaftor/ivacaftor (TRIKAFTA®) or newly developed therapies as they are developed. In some such embodiments, such medications include, without limitation, at least one of ivacaftor, lumacaftor/ivacaftor (ORKAMBI®), and tezacaftor/ivacaftor (SYMDEKO®), and elexacaftor/tezacaftor/ivacaftor (TRIKAFTA®).

### H. Bronchodilator/Inhaled Medications

In some embodiments, the therapeutic intervention comprises administering one or more inhaled medications for treating cystic fibrosis or cystic fibrosis-associated clinical outcomes. In some embodiments, the inhaled medications include a bronchodilator that helps relax the muscles around the bronchial tubes may be employed. Such inhaled medications include, without limitation, at least one of albuterol or levabuterol.

In some embodiments, the inhaled medications include one or more of the aforementioned probiotics and antibiotics (e.g., inhaled antipseudomonal antibiotics). In some embodiments, such inhaled medications include inhaled steroidal anti-inflammatory medications, such as beclomethasone dipropionate (Qvar®), budesonide (Pulmicort®), budesonide/formoterol (Symbicort®), fluticasone (Flovent®), fluticasone inhaled powder (Arnuity® Ellipta®), fluticasone/salmeterol (Advair®), mometasone (Asmanex®), and mometasone/formoterol (Dulera®).

### I. PANCREATIC ENZYME REPLACEMENT

In some embodiments, the therapeutic intervention includes pancreatic enzyme replacement therapy (PERT), including an adjustment to PERT already administered to an individual. The majority of individuals with cystic fibrosis suffer from pancreatic insufficiency where the individuals suffer from a lack of digestive enzymes made by their pancreas. In some embodiments, medications for exocrine pancreatic insufficiency, or pancreatic enzyme replacement therapy (PERT), may be employed. Pancreatic enzyme replacement therapy (PERT) involves taking digestive enzymes that assist the digestion of fat, carbohydrates and proteins-pancreatic enzyme products (PEPs).

Pancreatic enzyme products (PEPs) generally contain a mixture of the digestive enzymes amylase, lipase, and protease. In some embodiments, the medications for pancreatic enzyme replacement include, without limitation, pancreatin, pancrelipase, or other pancreatic enzyme substitute. In some embodiments, the medications for pancreatic enzyme replacement include, without limitation, Creon®, Zenpep®, Pancreaze®, Ultresa®, Viokace®, Pertzye®, Nutrizym®, Pancrease®, and Pancrex®.

### J. Physical Therapies/Other Procedures

In some embodiments, the therapeutic intervention comprises providing physical therapies and other medical procedures to the patient, for treating cystic fibrosis or cystic fibrosis-associated clinical outcomes in a subject in need thereof.

The physical therapy may include prescribing postural drainage, chest physical therapy, or pulmonary rehabilitation. The postural drainage may be performed by getting into positions that make it easier for mucus to drain from the lungs. The chest physical therapy may include, without limitation, at least one of vibrating vest and chest wall oscillation. The pulmonary rehabilitation may include, without limitation, at least one of physical exercise, breathing techniques, and nutritional counseling.

In some embodiments, the therapeutic intervention comprises administering a fecal microbiota transplantation (FMT) to the patient. FMT comprises the introduction of stools from a healthy donor into the digestive tract of a patient, in order to restabilize the intestinal microbiota of the patient. This fecal microbiota transplant may be allogenic (from a healthy donor individual to a patient) or autologous (from an individual to herself or himself). In some embodiments, the fecal microbiota transplant comprises one or more bacteria from the genera/family Bacteroides, Bifidobacterium, Faecalibacterium, Clostridiales, Roseburia, Ruminococcaceae, Eubacterium, Streptococcus and Ruminococcus.

### IV. EXEMPLARY EMBODIMENTS

### Exemplary Embodiment A

A1: In one aspect, the present disclosure provides a method for reducing frequency and/or number of upper respiratory infections in a patient with cystic fibrosis. The method comprises identifying a patient with cystic fibrosis (CF) as having a high risk for upper respiratory infections (URIs) based on the patient's stool microbiota, and providing a therapeutic intervention to the patient. A2: In certain embodiments of A1, the patient is identified as having a high risk for frequent URIs. A3: In certain embodiments of A1, the patient is identified as having a high risk for numerous URIs. A4: In certain embodiments of A1, the patient is identified as having a high risk for more than one URI per year. A5: In certain embodiments of A1-A4, the high risk is determined based upon a relative abundance of one or more genera of stool microbiota. A6: In certain embodiments of A1-A5, the patient is identified as having a high risk based upon a relative abundance of one or more of Faecalibacterium, Butyricoccus, or Bacteroides. A7: In certain embodiments of A1-A5, the patient's risk classification is determined based upon relative abundance of genera of stool microbiota, wherein said genera include at least 2, at least 3, at least 4, at least 5, at least 6, at least 7, at least 8, or at least 9 of the following genera: Faecalibacterium, Butyricicoccus, Bacteroides, Lachnospiraceae, Dielma, Sutterella, Faecalicoccus, Dorea, Staphylococcus, and Prevotella, A8: In certain embodiments of A1-A5, the patient's risk classification is determined based upon relative abundance of genera of stool microbiota, wherein said genera include all of the following genera: Faecalibacterium, Butyricicoccus, Bacteroides, Lachnospiraceae, Dielma, Sutterella, Faecalicoccus, Dorea, Staphylococcus, and Prevotella,

### Exemplary Embodiment B

B1: In one aspect, the present disclosure provides a method for reducing systemic inflammation in a patient with cystic fibrosis (CF) based on the patient's stool microbiota. The method comprises identifying the patient as having systemic inflammation based on the patient's stool microbiota, and providing a therapeutic intervention to the patient. B2: In certain embodiments of B1, the patient is identified as having systemic inflammation based upon a relative abundance of one or more genera of stool microbiota. B3: In certain embodiments of B1-B2, the patient is identified as having systemic inflammation based upon a relative abundance of one or more of Lactococcus, Kluyvera, or Klebsiella. B4: In certain embodiments of B1-B2, the patient's risk classification is determined based upon relative abundance of genera of stool microbiota, wherein said genera include at least 2, at least 3, at least 4, at least 5, at least 6, at least 7, at least 8, or at least 9 of the following genera: Lactococcus, Kluyvera, Klebsiella, Bilophila, Eubacterium, Corynebacterium, Flavonifractor, Bifidobacterium, Terrisporobacter, and Anaerococcus. B5: In certain embodiments of B1-B2, the patient's risk classification is determined based upon relative abundance of genera of stool microbiota, wherein said genera include all of the following genera: Lactococcus, Kluyvera, Klebsiella, Bilophila, Eubacterium, Corynebacterium, Flavonifractor, Bifidobacterium, Terrisporobacter, and Anaerococcus

### Embodiment C

C1: In certain embodiments of A1-A8 or B1-B5, the therapeutic intervention comprises one or more of (i) at least one bacterial composition; (ii) at least one probiotic; (iii) at least one prebiotic; (iv) at least one antibiotic; (v) at least one anti-inflammatory medication; (vi) at least one mucus-thinning drug; (vii) at least one cystic fibrosis transmembrane conductance regulator (CFTR) function-improving medication; (viii) at least one bronchodilator or inhaled medication; and/or (ix) at least one pancreatic enzyme replacement therapy. C2: In certain embodiments of C1, the therapeutic intervention comprises altering the intestinal microbiome. C3: In certain embodiments of C1 or C2, the therapeutic intervention comprises reducing the risk of, severity of, or delaying the onset of P. aeruginosa infection or chronic colonization. C4: In certain embodiments of C1-C3, the therapeutic intervention comprises reducing the risk of, severity of, or delaying cystic fibrosis-associated outcomes. C5: In certain embodiments of C4, the cystic fibrosis-associated outcomes includes at least one of pulmonary exacerbation, infection or chronic colonization with P. aeruginosa or other pathogen, growth failure or diagnosis of failure to thrive, early all-cause hospitalization, nutritional malabsorption, gastrointestinal-related outcomes (including but not limited to small bowel bacterial overgrowth, decreased absorption of essential fats), and disease-associated changes in body mass index (BMI). C6: In certain embodiments of C1, the at least one pancreatic enzyme replacement product comprises pancreatin, pancrelipase, or other pancreatic enzyme substitutes. C7: In certain embodiments of C1, the at least one probiotic comprises a microbial composition comprising at least one of Bifidobacterium, Bacteroides, Faecalibacterium, Clostridiales, Roseburia, Ruminococcaceae, Eubacterium, Streptococcus, and Ruminococcus. C8: In certain embodiments of C1, the at least one prebiotic comprises at least one of oligosaccharides, inulin, fructooligosaccharide (FOS), galactooligosaccharide (GOS), palatinoseoligosaccharide, soybean oligosaccharide, gentiooligosaccharide, xylooligomers, non-degradable starch, lactosaccharose, lactulose, lactitol, maltitol, polydextrose, and pectin. C9: In certain embodiments of C1, the method further comprises prescribing postural drainage, chest physical therapy, and/or pulmonary rehabilitation. C10: In certain embodiments of C9, the chest physical therapy comprises at least one of vibrating vest and chest wall oscillation. C11: In certain embodiments of C9, the pulmonary rehabilitation includes at least one of physical exercise, breathing techniques, and nutritional counseling. C12: In certain embodiments of C1, the at least one antibiotic is chosen from at least one of penicillin (methicillin, oxacillin, naficillin, cabencillin, amoxicillin, clavulanic acid, cloxacillin, dicloxacillin, ticarcillin, piperacillin, mezlocillin, azlocillin, tazobactam); cephalosporins (cephalexin, cefdinir, cefprozil, ceflacor, cefuroxime, cefepime); sulfa antibiotics (sulfamethoxazole, trimethoprim); aminoglycosides (tobramycin, amikacin, gentamicin); erythromycin/sulfisoxazole; macrolides (erythromycin, clarithromycin, azithromycin); tetracyclines (tetracycline, doxycycline, minocycline, tigecycline); vancomycin; imipenem; meripenem; colistimethate); quinolones (ciprofloxacin, levofloxacin); aztreonam; linezolid. C13: In certain embodiments of C1, the at least one anti-inflammatory medication is chosen from at least one of corticosteroid, ibuprofen, non-steroidal anti-inflammatory medication, anti-inflammatory cytokines, antibody or antigen binding fragment thereof to pro-inflammatory cytokine, antioxidants, protease inhibitors. C14: In certain embodiments of C1, the at least one cystic fibrosis transmembrane conductance regulator (CFTR)-improving medication is at least one of ivacaftor, lumacaftor/ivacaftor (ORKAMBI®), and tezacaftor/ivacaftor (SYMDEKO®), and elexacaftor/tezacaftor/ivacaftor (TRIKAFTA®). C15: In certain embodiments of C1, the at least one mucus-thinning drug is chosen from at least one of hypertonic saline, dornase alfa, and acetylcysteine. C16: In certain embodiments of C1, the at least one bronchodilator comprises albuterol or levabuterol.

### Embodiment D

D1: In certain embodiments of A1-A8, B1-B5, or C1-C16, the therapeutic intervention comprises altering the intestinal microbiome. D2: In certain embodiments of D1, the method comprises administering both Bifidobacterium and Bacteroides. D3: In certain embodiments of D1 or D2, the method further comprises administering Streptococcus. D4: In certain embodiments of D1-D3, altering the intestinal microbiome treats cystic fibrosis or alleviates a symptom thereof. D5: In certain embodiments of D1-D4, altering the intestinal microbiome comprises reducing the risk of, severity of, or delaying the onset of pathogenic microbial infection, wherein the pathogenic microbial infection comprises Staphylococcus aureus infection, Pseudomonas aeruginosa infection, Stenotrophomonas spp. infection, Haemophilus influenzae infection, nontuberculous mycobacterial (including but not limited to Mycobacteria abscessus or Mycobacteria avium) infection, Burkholderia cepacia complex infection, viral or fungal infection, or co-infections with multiple pathogens. D6: In certain embodiments of D1-D5, altering the intestinal microbiome comprises reducing the risk of, severity of, or delaying the onset of P. aeruginosa infection or chronic colonization. D7: In certain embodiments of D1-D6, altering the intestinal microbiome comprises reducing the risk of, severity of, or delaying pulmonary exacerbation. D8: In certain embodiments of D4, treating cystic fibrosis comprises reducing the risk of, severity of, or delaying cystic fibrosis-associated outcomes.

### Embodiment E

E1: In certain embodiments of A1-A8 or B1-B5, C1-C16, or D1-D8, the patient is 0 days old to 3 years old, 0 days old to 2 years old, 0 days old to 18 months old, 1 week old to 12 months old, 3 months old to 9 months old, or 1 week old to 6 months old. E2: In certain embodiments of E1, the patient is 0 days old to 12 months old, 1 week old to 12 months old, 3 months old to 9 months old, or 1 week old to 6 months old. E3: In certain embodiments of A1-A8 or B1-B5, C1-C16, or D1-D8, the patient is a pediatric patient. E4: In certain embodiments of E3, the subject and/or the patient is 1 week old to 6 months old. E5: In certain embodiments of E3, the subject and/or the patient is 1 week old to 12 months old. E6: In certain embodiments of E3, the subject and/or the patient is 1 week old to three years old. E7: In certain embodiments of E3, the subject and/or the patient is 1 week old to four years old.

### Exemplary Embodiment F

F1: In one aspect, the present disclosure provides a computer-implemented system for identifying a patient with cystic fibrosis as having a high risk for upper respiratory infections based on the patient's stool microbiota. The computer-implemented system comprises a machine learning model trained with data comprising (a) risk classifications for subjects with cystic fibrosis (CF), and (b) stool microbiota information for each of the subjects. The subjects are classified as having (i) a high risk of upper respiratory infections (URIs), or (ii) a low risk of URIs and/or a medium risk of URIs. The trained machine learning model is configured to analyze a patient's stool microbiota information as input values, and to provide the patient's risk classification as an output value. F2: In certain embodiments of F1, the subject is classified as high risk when the subject has a URI frequency of more than 1 per year. F3: In certain embodiments of F1, the subject is classified as high risk when the subject has 2 or more URIs between 0 days to 2 years old. F4: In certain embodiments of F1-F3, the patient's risk classification is determined based upon a relative abundance of said one or more genera of stool microbiota. F5: In certain embodiments of F1-F4, the patient's risk classification is determined based upon a relative abundance of one or more of Faecalibacterium, Butyricoccus, or Bacteroides. F6: In certain embodiments of F1-F4, the patient's risk classification is determined based upon relative abundance of genera of stool microbiota, wherein said genera include at least 2, at least 3, at least 4, at least 5, at least 6, at least 7, at least 8, or at least 9 of the following genera: Faecalibacterium, Butyricicoccus, Bacteroides, Lachnospiraceae, Dielma, Sutterella, Faecalicoccus, Dorea, Staphylococcus, and Prevotella, F7: In certain embodiments of F1-F4, the patient's risk classification is determined based upon relative abundance of genera of stool microbiota, wherein said genera include all of the following genera: Faecalibacterium, Butyricicoccus, Bacteroides, Lachnospiraceae, Dielma, Sutterella, Faecalicoccus, Dorea, Staphylococcus, and Prevotella, F8: In certain embodiments of F1-F7, the trained machine learning model is configured to receive a patient's relative abundance of genera of the stool microbiota as input values. F9: In certain embodiments of F1-F8, the trained machine learning model is configured to receive amplicon sequence variant (ASV) regions.

### Exemplary Embodiment G

G1: In one aspect, the present disclosure provides a computer-implemented system for identifying a patient with cystic fibrosis as having systemic inflammation based on the patient's stool microbiota. The computer-implemented system comprises a machine learning model trained with data comprising (a) risk classifications for subjects with cystic fibrosis (CF), and (b) stool microbiota information for each of the subjects. The subjects are classified as having (i) high systemic inflammation, or (ii) medium systemic inflammation and/or low systemic inflammation. The trained machine learning model is configured to analyze a patient's stool microbiota information as input values, and to provide the patient's systemic inflammation as an output value. G2: In certain embodiments of G1, the subjects are classified based on neutrophil-to-lymphocyte ratio (NLR) as a biomarker. G3: In certain embodiments of G1-G2, the patient's risk classification is determined based upon a relative abundance of one or more genera of stool microbiota. G4: In certain embodiments of G1-G3, the patient's risk classification is determined based upon a relative abundance of one or more of Lactococcus, Kluyvera, or Klebsiella. G5: In certain embodiments of G1-G3, the patient's risk classification is determined based upon relative abundance of genera of stool microbiota, wherein said genera include at least 2, at least 3, at least 4, at least 5, at least 6, at least 7, at least 8, or at least 9 of the following genera: Lactococcus, Kluyvera, Klebsiella, Bilophila, Eubacterium, Corynebacterium, Flavonifractor, Bifidobacterium, Terrisporobacter, and Anaerococcus. G6: In certain embodiments of G1-G3, the patient's risk classification is determined based upon relative abundance of genera of stool microbiota, wherein said genera include all of the following genera: Lactococcus, Kluyvera, Klebsiella, Bilophila, Eubacterium, Corynebacterium, Flavonifractor, Bifidobacterium, Terrisporobacter, and Anaerococcus. G7: In certain embodiments of G1-G6, the trained machine learning model is configured to receive a patient's relative abundance of genera as input values. G8: In certain embodiments of G1-G7, the trained machine learning model is configured to receive amplicon sequence variant (ASV) regions.

### Exemplary Embodiment H

H1: In certain embodiments of F1-9 or G1-G8, the subject and/or the patient is a pediatric patient. H2: In certain embodiments of H1, the subject and/or the patient is 1 week old to 6 months old. H3: In certain embodiments of H1, the subject and/or the patient is 1 week old to 12 months old. H4: In certain embodiments of H1, the subject and/or the patient is 1 week old to three years old. H5: In certain embodiments of H1, the subject and/or the patient is 1 week old to four years old. H6: In certain embodiments of F1-9 or G1-G8, the subject and/or the patient is 0 days old to 3 years old, 0 days old to 2 years old, 0 days old to 18 months old, 1 week old to 12 months old, 3 months old to 9 months old, or 1 week old to 6 months old. H7: In certain embodiments of F1-9 or G1-G8, the subject and/or the patient is 0 days old to 12 months old, 1 week old to 12 months old, 3 months old to 9 months old, or 1 week old to 6 months old.

### V. DEFINITIONS AND SUPPORTING INFORMATION

The terms “gut microbiota” or “gut microbiome” or “intestinal microbiota” or “intestinal microbiome” are used interchangeably and refer to the microorganisms that colonize the gastrointestinal tract of a human. As used herein, the terms “microbe” or “microorganism” encompass both prokaryotic organisms including bacteria and archaea, and eukaryotic organisms, including fungi, other single-celled eukaryotes, and viruses, present in mammalian microbiota.

By “modifying the intestinal microbiome”, “altering the intestinal microbiome”, “improving the gut microbiome” or “improving the intestinal microbiome” is meant any change, either increase or decrease, of the intestinal microbiota or microbiome in a subject. In some embodiments, modifying, altering, improving the intestinal microbiota includes increasing or decreasing the levels of specific bacteria, such as in the gastrointestinal tract of a subject. In some embodiments, modifying, altering, improving the intestinal microbiota includes increasing the levels of the bacteria described herein in the gastrointestinal tract of a subject.

By “increase,” “increasing”, “decrease” or “decreasing” is meant a change in the levels of specific bacteria in the gastrointestinal tract of a subject. An increase or decrease may include a change of any value from 10% and 100%, or of any value from 30% and 60%, or over 100%, for example, a change of about 10%, 20% 30%, 40%, 50%, 60%, 70%, 80%, 90%, 95% or more, when compared to a control. In some embodiments, the increase or decrease may be a change of about 1-fold, 2-fold, 5-fold, 10-fold, 100-fold, or more, when compared to a control.

Specific taxa and changes in microbiota discussed herein can be detected using various methods, including without limitation quantitative PCR (qPCR) or high-throughput sequencing (e.g., shotgun metagenome sequencing) methods which detect over- and under-represented genes in the total bacterial population (e.g., screening of microbial 16S ribosomal RNAs (16S rRNA)), or transcriptomic or proteomic studies that identify lost or gained microbial transcripts or proteins within total bacterial populations, or metabolomics, as previously described (e.g., Madan et al., mBio 2012; Hoen et al., J Pediatr, 2015; Filkins et al., J Bacteriol, 2012; Price et al., Microbiome, 2013; Gifford et al., J Cyst Fibros, 2014; Hampton et al., Microbiome, 2014).

As used herein, the term “16S rRNA sequencing” refers to the sequencing of 16S ribosomal RNA (rRNA) gene sequences by using primers such as universal primers and/or species-specific primers to identify the bacteria present in a sample. 16S rRNA genes contain both highly conserved sites and hypervariable regions that can provide species-specific signature sequences useful for identification of bacteria. Such universal primers are well known in the art.

As used herein, the term “operational taxonomic unit” or “OTU” refers to classification of microbes within the same, or different, OTUs using techniques, as described herein, or known in the art. OTU refers to a terminal leaf in a phylogenetic tree and is defined by a nucleic acid sequence, e.g., the entire genome, or a specific genetic sequence, and all sequences that share sequence identity to this nucleic acid sequence at the level of species. As used herein, the term “amplicon sequence variant” or “ASV” refers to classification using amplicon reads following the removal of erroneous sequences generated during amplification and sequencing, making it possible to distinguish sequence variation by a single nucleotide difference.

As used herein, “cystic fibrosis-associated clinical outcomes” or “disease progression in cystic fibrosis” may include pulmonary exacerbation, infection or chronic colonization with P. aeruginosa or other pathogen, growth failure or diagnosis of failure to thrive, early all-cause hospitalization, nutritional malabsorption, gastrointestinal-related outcomes such as small bowel bacterial overgrowth, decreased absorption of essential fats, and/or disease-associated changes in body mass index (BMI).

As used herein, the term “pulmonary exacerbation” generally entails acute worsening of symptoms (e.g., increased cough, sputum production, shortness of breath) accompanied by an acute decrease in lung function and often resulting in therapeutic interventions including antibiotics and/or hospitalization.

The terms “treatment,” “treating” or “therapy” encompass prophylactic, palliative, therapeutic, and nutritional modalities of administration of the bacterial compositions described herein. Accordingly, treatment includes amelioration, alleviation, reversal, or complete elimination of one or more of the symptoms in a subject diagnosed with, or known to have, cystic fibrosis, or cystic fibrosis-associated clinical outcomes (as discussed in the definition of “cystic fibrosis-associated clinical outcomes” above including, but not limited to, pulmonary exacerbation, or P. aeruginosa infection), or be considered to derive benefit from the alteration of intestinal microbiota. Such treatment also includes treating gastrointestinal (GI) outcomes such as prevention of small bowel bacterial overgrowth, improvement in absorption of essential fats, decrease in growth failure.

In the context of the present application, a “treatment” is a procedure which alleviates or reduces the negative consequences of cystic fibrosis. Any treatments or potential treatments can be used in the context herein. A treatment is not necessarily curative, and may reduce the symptom or effect of cystic fibrosis by a certain percentage over an untreated subject. The percentage reduction or diminution can be from 10% up to 20, 30, 40, 50, 60, 70, 80, 90, 95, 99 or 100%. “Treatment” also includes methods or preventing, inhibiting the development, or reducing the risk of cystic fibrosis, unless otherwise stated. It will be appreciated that, although not precluded, treating cystic fibrosis or the risk of developing cystic fibrosis does not require that the disease or the risk be completely eliminated.

As used herein, a “therapeutically effective amount” of an agent refers to an amount effective, at dosages and for periods of time necessary, to achieve the desired therapeutic result, such as treatment, prevention, or amelioration of cystic fibrosis, upper respiratory infection or risk thereof, systemic inflammation, or other cystic fibrosis-associated clinical outcomes. A therapeutically effective amount may vary according to factors such as the disease state, age, sex, and weight of the subject, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects. Ultimately, the prescribers or researchers will decide the appropriate amount and dosage regimen.

As used herein, “inhibiting the development of,” “reducing the risk of,” “prevent,” “preventing,” and the like refer to reducing the probability of developing a symptom, condition, or disorder in a patient who may not yet have a symptom, condition, or disorder, but may have a genetic predisposition to developing it. For example, “preventing” includes inhibiting or reducing the probability of developing pulmonary exacerbation, or P. aeruginosa infection or chronic colonization, or any other cystic fibrosis-associated clinical outcomes. As used herein, “at risk,” “susceptible to,” or “having a genetic predisposition to,” refers to having a propensity to develop a symptom, condition, or disorder. For example, a patient may have been diagnosed as having cystic fibrosis, but may not yet have respiratory exacerbation or infection with P. aeruginosa.

As used herein “patient” or “subject” refers to any human being receiving or who may receive a therapeutic intervention; as indicated above, “subject” usually refers to an individual whose data is used for the training of a machine learning model, while “patient” usually refers to an individual to be identified using the present methods and systems as having a high risk of upper respiratory infection or as having systemic inflammation. These terms also include mammals. Mammals include, but are not limited to, domesticated animals (e.g., cows, sheep, cats, dogs, and horses), primates (e.g., humans and non-human primates such as monkeys), rabbits, and rodents (e.g., mice and rats). In some embodiments, the subject is a patient. In some embodiments, the subject may be a human infant with a family history of cystic fibrosis. The subject may be an infant, such as a human infant one year old or less, or three months old or less. In some embodiments, the subject may be a human infant at any age from 1 day to 350 days old, such as 1 day, 10 days, 20 days, 30 days, 40 days, 50 days, 60 days, 70 days, 80 days, 90 days, 100 days, 110 days, 120 days, 130 days, 140 days, 150 days, 160 days, 170 days, 180 days, 190 days, 200 days, 210 days, 220 days, 230 days, 240 days, 250 days, 260 days, 270 days, 280 days, 290 days, 300 days, 310 days, 320 days, 330 days, 340 days, 350, 365 days old. In some embodiments, the subject may be a human infant of 0 days old to 3 years old, 0 days old to 2 years old, 0 days old to 18 months old, 1 week old to 12 months old, 1 week old to 6 months old.

The subject may be suspected of having or at risk for cystic fibrosis-associated clinical outcomes (including pulmonary exacerbation, or P. aeruginosa and other pathogenic microbial infection or chronic colonization), be diagnosed with such cystic fibrosis-associated clinical outcomes, or be a control subject that is confirmed to not have any cystic fibrosis-associated clinical outcomes. Diagnostic methods for the cystic fibrosis-associated clinical outcomes, and the clinical delineation of such diagnoses are known to those of ordinary skill in the art.

The term “about” means a numeric value, including, for example, whole numbers, fractions, and percentages, whether or not explicitly indicated. The term “about” refers generally to a range of numerical values (e.g., ±5 to 10% of the recited range) that one of ordinary skill in the art would consider equivalent to the recited range (e.g., having the same function or result). When terms such as “at least” and “about” precede a list of numerical values or ranges, the terms modify all of the values or ranges provided in the list. In some instances, the term “about” may include numerical values that are rounded to the nearest significant figure.

As used herein “diagnosis” or “identifying a patient having” refers to a process of determining if an individual is afflicted with, or has a predisposition or is at risk to develop, a condition, disorder, or symptom, e.g., such as pulmonary exacerbation, cystic fibrosis-associated clinical outcomes, P. aeruginosa infection or colonization, and/or other condition, disorder, or symptom associated with worsening disease progression and/or earlier mortality.

Although certain embodiments and advantages have been described in detail, it should be understood that various changes, substitutions and alterations can be made herein without departing from the spirit and scope as defined in the appended claims.

Embodiments will be further illustrated in the following Examples which are given for illustration purposes only and are not intended to limit the application in any way.

### EXAMPLES

The following methods and materials were used in the following examples.

### A. Microbial Sequencing of Samples

Stool in diapers was collected by parents or guardians of cwCF and stored in home freezers; upon arrival in the lab, these diaper samples were stored frozen at −80° C. until processing, consistent with procedures reported previously (See Hoen et al., 2015.) All DNA was extracted from 100 mg of thawed stool sample using the Zymo Quick-DNA Fecal Microbe miniprep kit (Cat. No. D6010) as per the manufacturer's instructions. Illumina targeted sequencing of the V4-V5 region of the 16S rRNA gene was performed at the Marine Biological Laboratory in Woods Hole, MA. Adapter sequences were removed using cutadapt (v 2.4). All samples were further processed through the DADA2(v 1.22.0) pipeline for quality filtering and trimming (trimmed at 220 and 220 for forward and reverse reads, respectively) and merging of paired end reads to produce an amplicon sequence variant (ASV) table. ASVs were classified with taxa from the SILVA database (vSILVA_SSU_r138_2019) using the DECIPHER (v 2.22.0) pipeline. All sequence reads can be found in GenBank with CF cohort sequences found under accession number PRJNA170783 (SRP014429).

### B. Analysis of Microbial Populations in Stool

Code is available on GitHub at https://github.com/RebeccaAValls/RFM_CFinfants. Relative abundance, alpha diversity, and beta diversity measured were calculated and plotted for each sample using the package phyloseq (v1.38.0) and ggplot2 (v3.3.5). Each sample's ASV counts were normalized to percentage out of the sum of counts for that sample to obtain a relative abundance. The relative abundances of the top 10 genera for each classification (age, URIfreq, or NLR group) were calculated by averaging the relative abundance of all samples that belonged within that group. Phyloseq was used to plot alpha diversity metrics (Chao-1 and Shannon), and beta diversity calculated by Bray-Curtis distances. Analysis and permutation multivariate analysis of variance (ANOVA, PERMANOVA) with multiple comparisons were applied to determine statistical significance for alpha and beta diversity, respectively.

### C. Predictive Modeling of Clinical Data Using Bacterial Populations in Stool Samples

Random forest classification models were trained with the relative abundance of ASVs (at the genus level) from stool samples from cwCF. The R package randomForest (v 4.7.1) was used with ntree=10000, using default mtry of sqrt(p) where p is the number of input variables (or microbial genera) for building the tree. For the age prediction model of Example 1, samples were labeled with the age of the cwCF at which the sample was collected. Each sample was therefore classified as belonging to 1 of 5 groups: <1, 1, 2, 3, or 4 (Age (years)). 21 samples were randomly selected from each age group in order to train the age prediction model. For the URIfreq model of Example 3, the total URI per patient was calculated and normalized by age to determine URIfreq per patient. Each sample was then classified as coming from a patient with Low, Medium, or High URIfreq. For the NLR model of Example 4, a neutrophil to lymphocyte ratio was calculated by dividing absolute neutrophil counts by absolute lymphocyte counts. These values were then merged with stool samples from cwCF that were collected within the same 3-month period. Each sample was then classified as having a Low, Medium, or High NLR associated with the sample.

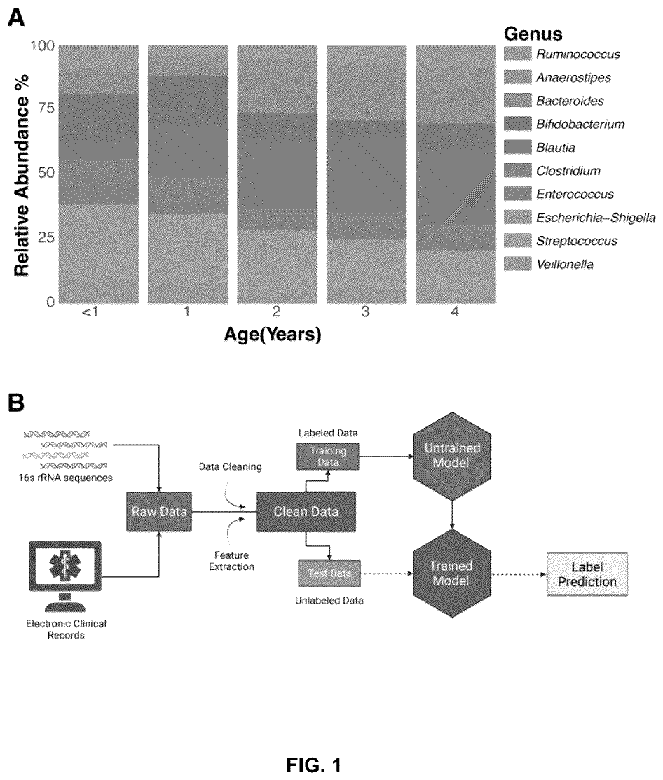

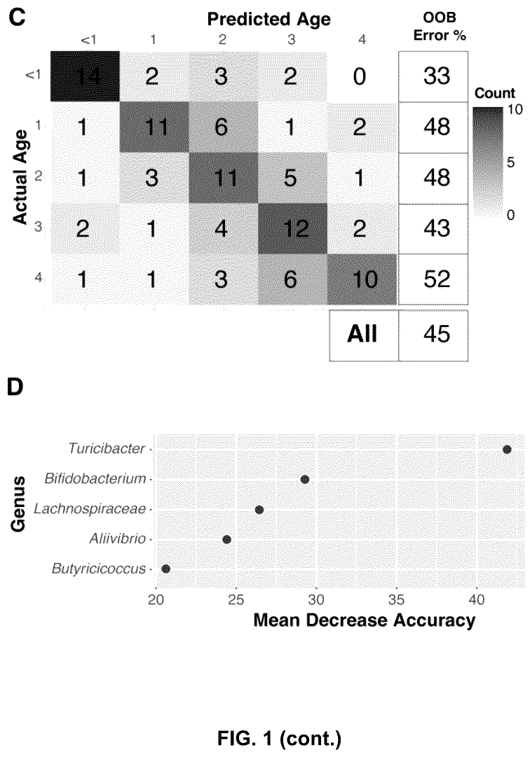

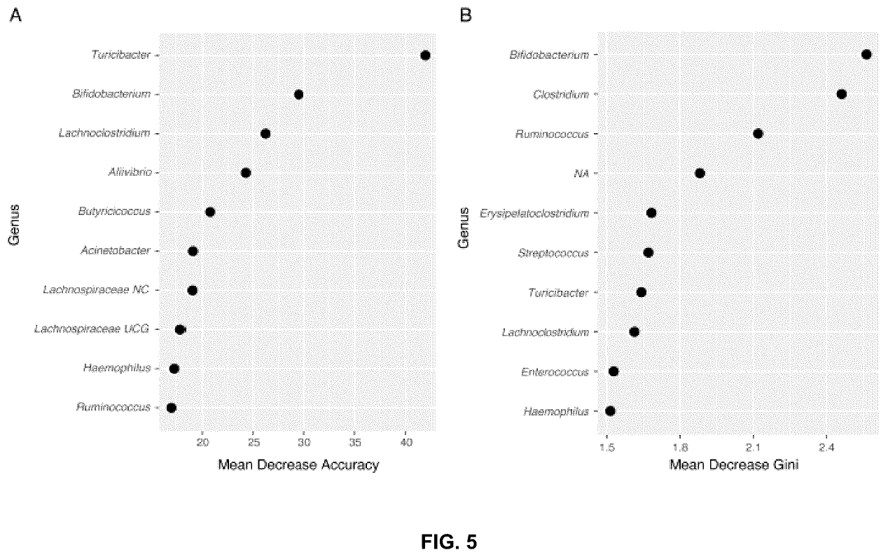

Model performance was evaluated based on the OOB per model. The taxa with the highest informative impact were identified by reviewing the random forest Mean Decrease Accuracy and Mean Decrease Gini values. The top 10 microbial taxa are listed in FIGS. 5-9, and subset to the top 5 microbial taxa in FIGS. 1-4 for simpler visualization and discussion.

### Example 1

This example demonstrates that random forest models can predict age of cwCF from stool microbiome composition. 282 stool samples were collected from 39 cwCF (Table 1) and 16S rRNA gene amplicon libraries were sequenced from these samples. To balance the samples per age group and to facilitate their training via a random forest model, samples were randomly subsetted into n=21 per age group. Relative abundances of stool microbiota were set as features for each sample. Each sample was further labeled with the age of cwCF at time of collection. The relative abundances of fecal microbiota were used to train a random forest model to predict the age category of cwCF (FIG. 1, panels A,B). The predictive power, or accuracy, of the model was determined from the out of bag score (OOB), a measure of how well a model predicted the age of a stool microbiome sample where age is not specified. When training a model with classified samples, our random forest model randomly selects 2/3 of all samples to build the decision trees that make the predictions, by default. The remaining samples (1/3) are not included in the training process. These out-of-bag (OOB) samples are used to validate the predictive power of the model. The OOB score reflects the number of correctly predicted sample classifications from the out of bag samples. In this case, the random forest predictive model correctly predicted age category of cwCF with 45% error (FIG. 1C), an error value higher than previously reported for infants without CF (Hayden et al., 2020). This decrease in prediction accuracy is not surprising, as several studies have shown that there is a marked dysbiosis in the gut of cwCF (Price et al., 2021; De Lisle et al., 2013; Hoffman et al., 2014; Kristensen et al., 2020; Meeker et al., 2020; van Dorst et al., 2022).

Model training was updated to make sure that patients which are used to train the model are not included in testing of the model. This prevents overfitting of the prediction power.

Additionally, Hayden et al., 2020 showed that cwCF have a delayed maturation of gut microbiota. Thus, the present data are consistent with previous findings in children, and indicate that the gut dysbiosis in CF may interfere with age-related community development in such a way that it makes predictions more difficult to make accurately.

The random forest model resulted in outputs for two measurements of importance. The Mean Decrease Accuracy, labeled as Impact Factor, lists the top features; in this case genera of stool bacteria, important for age-model output accuracy. For predicting age, these genera are Turicibacter, Bifidobacterium, and Lachnoclostridium. That is, if any of these genera were removed when generating the predictive model, the accuracy of the prediction would suffer. Although multiple iterations of the model resulted in a change in the order of the genera listed in panel D of FIG. 1, Turicibacter was consistently the top genus of bacteria driving the model output. A full list of the genera important for age-model output are shown in panel A of FIG. 5. The second measure of importance to the model, Mean Decrease Gini, is a measure of node purity. The greater a feature's Mean Decrease Gini, the better that genus is able to split classification groups into pure class nodes. For predicting age, Bifidobacterium, Clostridium, and Ruminococcus are the genera with highest node purity (panel B of FIG. 5).

### Example 2

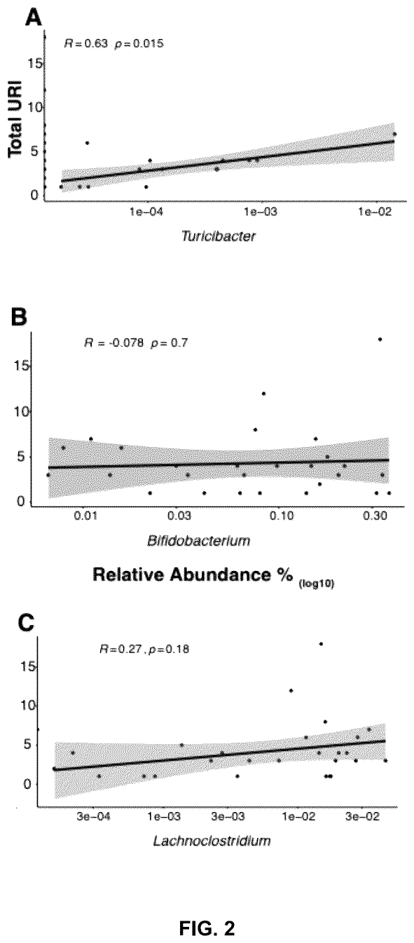

In this example, it was examined whether the genus Turicibacter, important for predicting age in cwCF, might inform other clinical outcomes. Specifically, it was examined whether the total number of URIs experienced by a patient would correlate with the relative abundance of Turicibacter, Bifidobacterium, and Lachnoclostridium in the stool across the first two years of life (panels A, B and C in FIG. 2). This example focused on the first two years of life because it is during these early years that the gut microbiome is establishing; the gut microbiota is considered more stable after ages 3-5 (see Price et al., 2021), although some studies suggest earlier stabilization, as early as 1-3 years (see Nielsen et al., 2015).

A modest, but statistically significant, positive correlation was found between total URIs and relative abundance of Turicibacter in stool (panel A of FIG. 2). Interestingly, 13 patients had no Turicibacter in the first two years of life. These 13 patients were further assessed for correlations between total URI and relative abundance of Bifidobacterium or Lachnoclostridium, the two other genera contributing to model accuracy, to determine if another top age predictor could be driving total URI in cwCF lacking Turicibacter; no such correlation was identified. No statistically significant correlation was identified between total URI and relative abundance of Bifidobacterium or Lachnoclostridium (see panels B and C in FIG. 2).

### Example 3

This example demonstrates that high URI frequency can be predicted for cwCF from stool microbiome composition. The modest correlation between Turicibacter and URI frequency suggested that directly applying random forests to the problem of predicting URI might better reveal specific taxa. Therefore, the frequency of upper respiratory infections (URIfreq) was calculated for each infant, that is, their total URI divided by age in years. The infants were then stratified into one of three URIfreq groups: Low (<1 URI per year of life), Medium (1 URI per year of life), and High (>1 URI per year of life). This stratification produced roughly equal numbers of cwCF in each group, as indicated in Table 1.

TABLE 1

| | | N of Classification Groups^{a} | | | | | |
|---|---|---|---|---|---|---|---|
| | | (patients/samples) | | | | | |
| | | <1 | 1 | 2 | 3 | 4 | Sum |
| Age | All | 27/44 | 32/89 | 25/71 | 23/57 | 19/21 | 39/282 |
| | Subset^{b} | 19/21 | 15/21 | 14/21 | 14/21 | 15/21 | 36/105 |

N of Classification Groups
(patients/samples)

| | Low | Medium | High | Sum |
|---|---|---|---|---|
| URIfreq | 8/73 | 8/68 | 12/105 | 28/246 |
| NLR | 6/11 | 8/11 | 7/11 | 14/33 |

^{a}Patient and sample n per classification group.
^{b}Subset of age indicates number of samples used to train the random forest model.

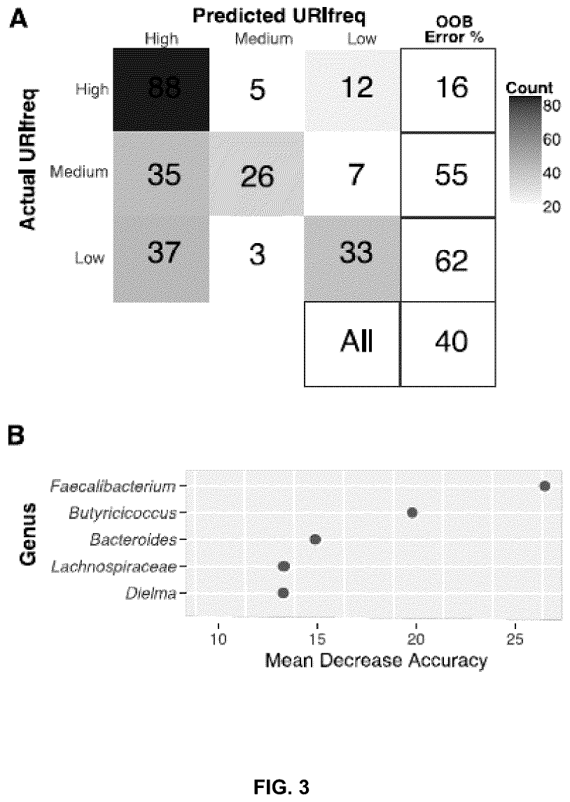

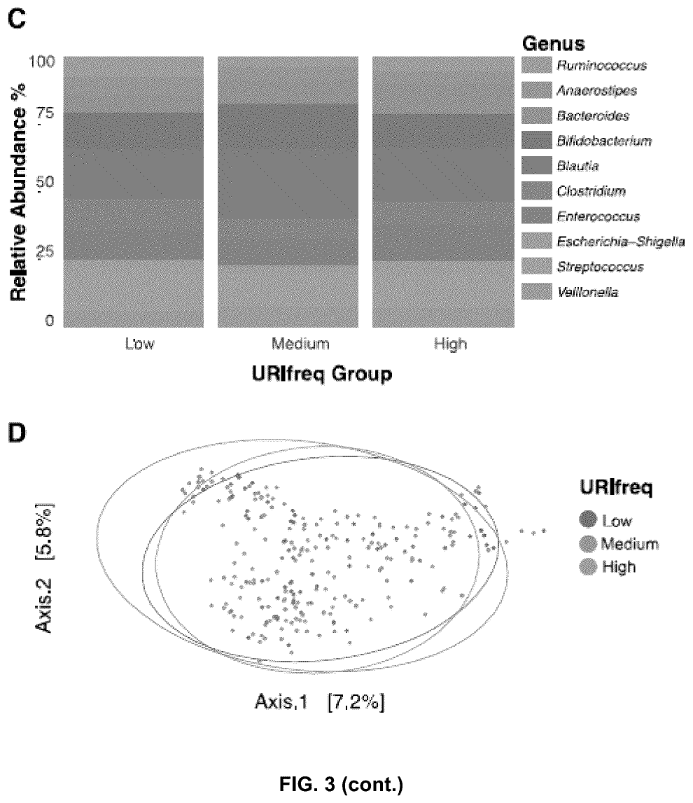

Although the URIfreq predictive model predicted URIfreq OOB with 40% error overall, the model predicting high URIfreq only had 16% error (panel A of FIG. 3). The top genera of stool bacteria important for URIfreq-model output are Faecalibacterium, Butyricicoccus and Bacteroides (panel B of FIG. 3). Table 2 shows values for pairwise comparison of beta diversity for URIfreq groups in FIG. 3.

TABLE 2

| pairs | Df | SumsOfSqs | F.Model | R2 | p.value | p.adjusted | sig |
|---|---|---|---|---|---|---|---|
| High vs Low | 1 | 0.7864209 | 1.957459 | 0.01099959 | 0.001 | 0.001 | ** |
| High vs Medium | 1 | 0.782652 | 1.948492 | 0.01126631 | 0.001 | 0.001 | ** |
| Low vs Medium | 1 | 0.7413204 | 1.851852 | 0.01314752 | 0.001 | 0.001 | ** |

In Tables 2-4, the column “sig”, short for significance, contains stars based on the p value. These are the associated Signif. codes for each p value: 0 ‘***’; 0.001 ‘**’; 0.01 ‘*’; 0.05 ‘⋅’; 0.1 ‘ ’, 1.

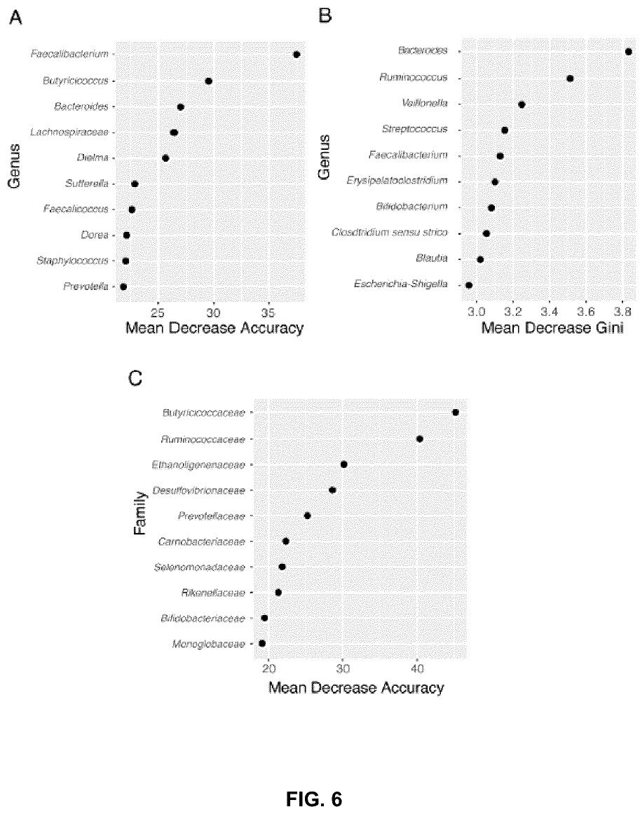

A full list of the genera important for URIfreq-model output are shown in panels A and B of FIG. 6. Faecalibacterium and Butyricicoccus secrete butyrate, a short chain fatty acid previously shown to reduce inflammation in the intestinal mucosa and be overall protective of gut physiology (Price et al., 2021; Yip et al., 2021; Jang et al., 2020). Plotting relative abundance of the 10 most abundant stool microbiota across URIfreq groups revealed that Bacteroides doubled in the High URIfreq group compared to the Low and Medium URIfreq groups (panel C of FIG. 3), a point addressed further herein. In order to determine whether a broader classification of microbiota could help inform function of microbes driving the predictive power of the URIfreq-model, the same model was assessed at a family level and it was found that the error did not change. The top family important for URIfreq prediction was Butyricicoccaceae (panel C of FIG. 6).

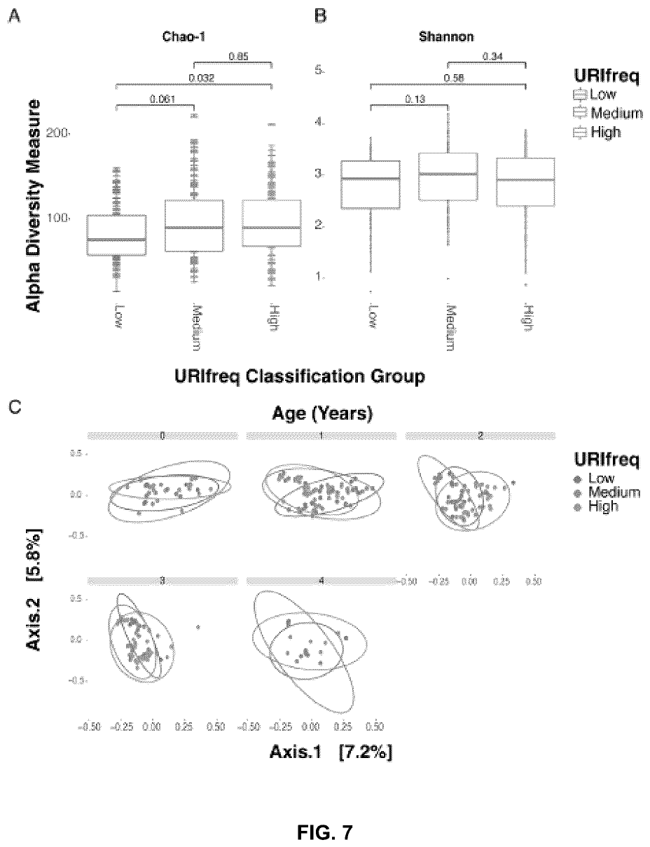

Next the microbial communities were examined of the children classified as Low, Medium or High in upper respiratory infection frequency. The Chao1-determined alpha diversity, a measure of microbial richness, showed a significant increase in the high URIfreq group (FIG. 7, panel A), but there was no significant increase in Shannon alpha diversity (FIG. 7, panel B). Table 3 shows values for pairwise comparison of beta diversity for URIfreq groups, per age group, in FIG. 7.

TABLE 3

| Bin_Year | pairs | Df | SumsOfSqs | F.Model | R2 | p.value | p.adjusted | sig |
|---|---|---|---|---|---|---|---|---|
| 0 | High vs Low | 1 | 0.7864209 | 1.957459 | 0.01099959 | 0.001 | 0.0015 | * |
| 0 | High vs Medium | 1 | 0.782652 | 1.948492 | 0.01126631 | 0.001 | 0.0015 | * |
| 0 | Low vs Medium | 1 | 0.7413204 | 1.851852 | 0.01314752 | 0.002 | 0.002 | * |
| 1 | High vs Low | 1 | 0.7864209 | 1.957459 | 0.01099959 | 0.001 | 0.0015 | * |
| 1 | High vs Medium | 1 | 0.782652 | 1.948492 | 0.01126631 | 0.001 | 0.0015 | * |
| 1 | Low vs Medium | 1 | 0.7413204 | 1.851852 | 0.01314752 | 0.004 | 0.004 | * |
| 2 | High vs Low | 1 | 0.7864209 | 1.957459 | 0.01099959 | 0.001 | 0.003 | * |
| 2 | High vs Medium | 1 | 0.782652 | 1.948492 | 0.01126631 | 0.002 | 0.003 | * |
| 2 | Low vs Medium | 1 | 0.7413204 | 1.851852 | 0.01314752 | 0.004 | 0.004 | * |
| 3 | High vs Low | 1 | 0.7864209 | 1.957459 | 0.01099959 | 0.002 | 0.002 | * |
| 3 | High vs Medium | 1 | 0.782652 | 1.948492 | 0.01126631 | 0.002 | 0.002 | * |
| 3 | Low vs Medium | 1 | 0.7413204 | 1.851852 | 0.01314752 | 0.002 | 0.002 | * |
| 4 | High vs Low | 1 | 0.7864209 | 1.957459 | 0.01099959 | 0.001 | 0.003 | * |
| 4 | High vs Medium | 1 | 0.782652 | 1.948492 | 0.01126631 | 0.002 | 0.003 | * |
| 4 | Low vs Medium | 1 | 0.7413204 | 1.851852 | 0.01314752 | 0.003 | 0.003 | * |

This finding indicates that sample richness was different between the URIfreq groups, but not evenness. Microbial communities in the stool from children with different rates of upper respiratory infection frequency were substantially similar in composition. Statistically, significant differences in community structure assessed by Bray-Curtis dissimilarity was revealed by PERMANOVA (adonis test) as a function of URIfreq group (P=0.001). However, differences were very small and account for less than 2% of the variability (R2=0.016) (panel D of FIG. 3). When the interaction of age is accounted for, composition of community structure remains similar. Although statistically significant, (P=0.009), accounting for URIfreq group and age explain little of the variability in composition (R2-0.011) as determine by PERMANOVA (FIG. 7, panel C). Collectively, these results indicate that the overall microbial composition of each URIfreq group are similar, but the differences can be predictive for URIfreq.

### Example 4

This example demonstrates that High NLR Classification can be predicted for cwCF from stool microbiome composition. Hematology data was obtained for a subset of cwCF in the cohort. Included in these hematology data were values for absolute neutrophil and lymphocyte levels. The ratio of absolute neutrophil count to absolute lymphocytes (NLR) provides a quantitative biomarker of systemic inflammation, where higher NLR has been shown to correlate with lower lung function (ppFEV₁) (see O'Brien C E, Price E T). The blood neutrophil to lymphocyte ratio correlates with clinical status in children with cystic fibrosis: A retrospective study. PLOS One. 2013; 8(10):1-7). Therefore, random forest models were created to identify features in the CF infant stool microbiome composition that predict NLR.

As before, the cwCF cohort was stratified into three groups: Low (0.2-0.46), Medium (0.47-0.85), or High (0.86-4) NLR, yielding an approximately equal number of individuals per group (see Table 1 above). Although the overall OOB error of the NLR predictive model was 64%, the model performed well at predicting high NLR with 27% OOB error (panel A of FIG. 4). Table 4 shows values for pairwise comparison of beta diversity for NLR groups in FIG. 4.

TABLE 4

| pairs | Df | SumsOfSqs | F.Model | R2 | p.value | p.adjusted | sig |
|---|---|---|---|---|---|---|---|
| Medium vs low | 1 | 0.4588526 | 1.187457 | 0.05604528 | 0.172 | 0.172 | not |
| | | | | | | | signif |
| Medium vs High | 1 | 0.6525143 | 1.638645 | 0.07572772 | 0.011 | 0.033 | not |
| | | | | | | | signif |
| low vs High | 1 | 0.5437049 | 1.367938 | 0.06401824 | 0.061 | 0.0915 | not |
| | | | | | | | signif |

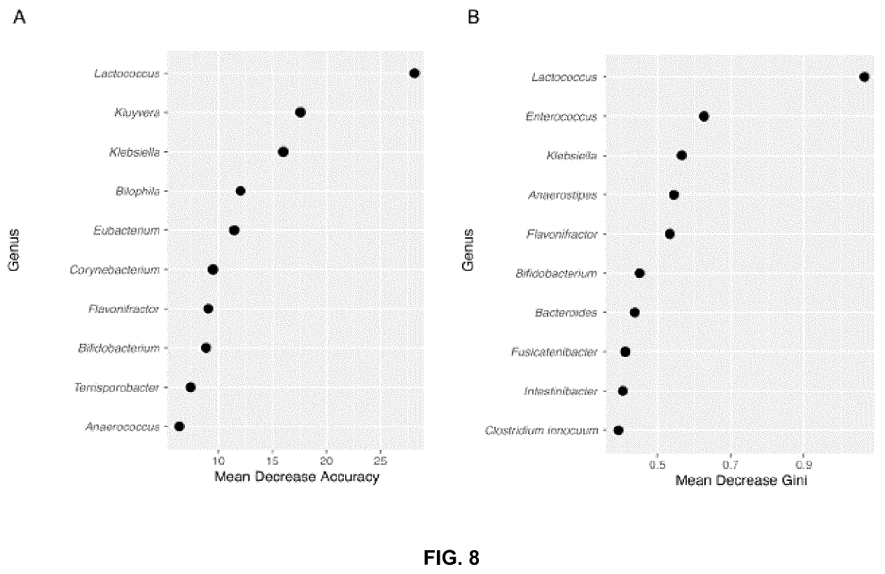

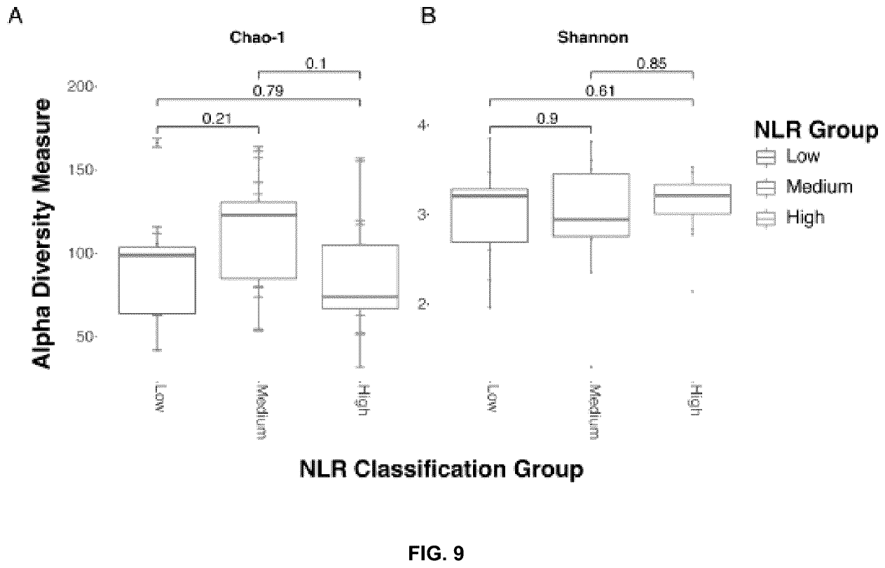

The top genera of stool bacteria important for NLR prediction were Lactococcus, Kluyvera, and Klebsiella (panel B of FIG. 4, panels A & B of FIG. 8). No significant difference was seen in the measure of alpha diversity (FIG. 9). Statistically different community structure assessed by Bray-Curtis dissimilarity was revealed by PERMANOVA (adonis test) as a function of NLR groups. However, again this difference was very small and accounts for less than 10% of the variability (R2=0.085) in beta diversity among NLR groups in infants with CF (panel D in FIG. 4).

There are several important implications of these findings. cwCF often experience chronic respiratory infections, leading to progressive, irreversible lung function decline and significant morbidity and premature mortality. Modulator therapy has revolutionized the treatment of eligible adults with CF. cwCF as young as age 6 are now eligible to receive modulator therapy, although by early childhood many children have already experienced pulmonary exacerbations. The present disclosure shows that for cwCF, stool microbiota composition is associated with higher upper respiratory infection frequency and increased systemic inflammation. These findings may aid in developing diagnostic tools that can allow physicians further understanding of which intestinal microbiota profiles are associated with health outcomes and to identify targets for preventative treatment for cwCF.

### EQUIVALENTS

The foregoing written specification is considered to be sufficient to enable one skilled in the art to practice the embodiments. The foregoing description and Examples detail certain embodiments and describes the best mode contemplated. It will be appreciated, however, that no matter how detailed the foregoing may appear in text, the embodiment may be practiced in many ways and should be construed in accordance with the appended claims and any equivalents thereof.

### REFERENCES

References discussed in the application, which are incorporated by reference in their entirety, for their intended purpose, which is clear based upon its context.
1. Price C E, O'Toole G A. The gut-lung axis in cystic fibrosis. J Bacteriol. 2021; 203(20:e00311-21).
2. De Lisle R C, Borowitz D. The cystic fibrosis intestine. Cold Spring Harb Perspect Med. 2013; 3(9):1-17.
3. Nielsen S, Needham B, Leach S T, et al. Disrupted progression of the intestinal microbiota with age in children with cystic fibrosis. Sci Rep. 2016; 6(October 2015):1-11.
4. Hoffman L R, Pope C E, Hayden H S, et al. Escherichia coli dysbiosis correlates with gastrointestinal dysfunction in children with cystic fibrosis. Clin Infect Dis. 2014; 58(3):396-399.
5. Ooi C Y, Syed S A, Rossi L, et al. Impact of CFTR modulation with Ivacaftor on gut microbiota and intestinal inflammation. Sci Rep. 2018; 8(1):1-8.
6. Kristensen M, Prevaes S M P J, Kalkman G, et al. Development of the gut microbiota in early life: The impact of cystic fibrosis and antibiotic treatment. J Cyst Fibros. 2020; 19(4): 553-561.
7. Antosca K M, Chernikova D A, Price C E, et al. Altered gut microbiota and intestinal inflammation. J Bacteriol. 2019; 201(16):1-15.
8. Hayden H S, Eng A, Pope C E, et al. Fecal dysbiosis in infants with cystic fibrosis is associated with early linear growth failure. Nat Med. 2020; 26(2):215-221.
9. Meeker S M, Mears K S, Sangwan N, et al. CFTR dysregulation drives active selection of the gut microbiome. PLOS Pathog. 2020; 16(1):1-17.
10. Yoon H Y, Kim H N, Lee S H, et al. Association between neutrophil-to-lymphocyte ratio and gut microbiota in a large population: a retrospective cross-sectional study. Sci Rep. 2018; 8(1):1-9.
11. Hoen A G, Li J, Moulton L A, et al. Associations between gut microbial colonization in early life and respiratory outcomes in cystic fibrosis. J Pediatr. 2015; 167(1): 138-147.e3.
12. Yip W, Hughes M R, Li Y, et al. Butyrate shapes immune cell fate and function in allergic asthma. Front Immunol. 2021; 12(February):1-13.
13. Stefan K L, Kim M V., Iwasaki A, Kasper D L. Commensal microbiota modulation of natural resistance to virus infection. Cell. 2020; 183(5): 1312-1324.e10.
14. Constantinides M G, Link V M, Tamoutounour S, et al. MAIT cells are imprinted by the microbiota in early life and promote tissue repair. Science (80-). 2019; 366(6464).
15. Valguarnera E, Wardenburg J B. Good gone bad: One toxin away from disease for Bacteroides fragilis. J Mol Biol. 2020; 432(4):765-785.
16. Cahenzli J, Köller Y, Wyss M, Geuking M B, McCoy K D. Intestinal microbial diversity during early-life colonization shapes long-term IgE levels. Cell Host Microbe. 2013; 14(5):559-570.
17. Al Nabhani Z, Dulauroy S, Marques R, et al. A weaning reaction to microbiota is required for resistance to immunopathologies in the adult. Immunity. 2019; 50(5):1276-1288.e5.
18. Depner M, Taft D H, Kirjavainen P V., et al. Maturation of the gut microbiome during the first year of life contributes to the protective farm effect on childhood asthma. Nat Med. 2020; 26(11):1766-1775.
19. Jang Y O, Lee S H, Choi J J, et al. Fecal microbial transplantation and a high fiber diet attenuates emphysema development by suppressing inflammation and apoptosis. Exp Mol Med. 2020; 52(7): 1128-1139.
20. Neri L D C L, Taminato M, Da Silva Filho L V R F. Systematic review of probiotics for cystic fibrosis patients: Moving forward. J Pediatr Gastroenterol Nutr. 2019; 68(3): 394-399.
21. van Dorst J M, Tam R Y, Ooi C Y. What do we know about the microbiome in cystic fibrosis? Is there a role for probiotics and prebiotics? Nutrients. 2022; 14(3): 1-27.
22. Wen L, Shi L, Kong X-L, et al. Gut microbiota protected against Pseudomonas aeruginosa pneumonia via restoring Treg/Th17 balance and metabolism. Front Cell Infect Microbiol. 2022; 12(June): 1-15.
23. Shah P, Kendall F, Khozin S, et al. Artificial intelligence and machine learning in clinical development: a translational perspective. NPJ Digit Med. 2019; 2(1).
24. Anahtar M N, Yang J H, Kanjilal S. Applications of machine learning to the problem of antimicrobial resistance: an emerging model for translational research. J Clin Microbiol. 2021; 59(7):e0126020.
25. AlJame M, Imtiaz A, Ahmad I, Mohammed A. Deep forest model for diagnosing COVID-19 from routine blood tests. Sci Rep. 2021; 11(1):1-12.
26. Price C E, Valls R A, Ramsey A R, et al. Intestinal Bacteroides modulates systemic inflammation and the microbial ecology in a mouse model of CF: Evidence for propionate and other short chain fatty acids reducing systemic inflammatory cytokines. bioRxiv. January 2022:2022.01.05.475125.
27. O'Brien C E, Price E T. The blood neutrophil to lymphocyte ratio correlates with clinical status in children with cystic fibrosis: A retrospective study. PLOS One. 2013; 8(10):1-7.
28. Bazett M, Bergeron M E, Haston C K. Streptomycin treatment alters the intestinal microbiome, pulmonary T cell profile and airway hyperresponsiveness in a cystic fibrosis mouse model. Sci Rep. 2016; 6(August 2015):1-13.
29. Li L, Fang Z, Liu X, et al. Lactobacillus reuteri attenuated allergic inflammation induced by HDM in the mouse and modulated gut microbes. PLOS One. 2020; 15(4): 1-14.
30. Ravaut M, Harish V, Sadeghi H, et al. Development and validation of a machine learning model using administrative health data to predict onset of Type 2 diabetes. JAMA Netw Open. 2021; 4(5):1-15.

### Exemplary Embodiments

Before various exemplary embodiments and examples are described, it is to be understood that the teachings of this disclosure are not limited to the particular embodiments described, and as such can, of course, vary.

Embodiment 1. A method of reducing frequency and/or number of upper respiratory infections in a patient with cystic fibrosis, the method comprising: identifying a patient with cystic fibrosis (CF) as having a high risk for upper respiratory infections (URIs) based on the patient's stool microbiota, and providing a therapeutic intervention to the patient.

Embodiment 2. The method of embodiment 1, wherein the patient is identified as having a high risk for frequent URIs.

Embodiment 3. The method of embodiment 1, wherein the patient is identified as having a high risk for numerous URIs.

Embodiment 4. The method of any of the foregoing embodiments, wherein the high risk is determined based upon a relative abundance of one or more genera of stool microbiota.

Embodiment 5. The method of any of the foregoing embodiments, wherein said one or more genera of stool microbiota comprises Faecalibacterium, Butyricoccus, or Bacteroides.

Embodiment 6. The method of any of the foregoing embodiments, wherein the patient is predicted as being at high risk of having more than one URI per year.

Embodiment 7. A method of reducing systemic inflammation in a patient with cystic fibrosis (CF) based on the patient's stool microbiota, the method comprising: identifying the patient as having systemic inflammation based on the patient's stool microbiota; providing a therapeutic intervention to the patient.

Embodiment 8. The method of embodiment 7, wherein the systemic inflammation is determined based upon a relative abundance of one or more genera of stool microbiota.

Embodiment 9. The method of embodiment 7, wherein said one or more genera of stool microbiota comprises Lactococcus, Kluyvera, or Klebsiella.

Embodiment 10. The method of any of the foregoing embodiments, wherein the method further comprises: collecting a stool sample from the patient; extracting microbiota nucleic acids from the stool sample; sequencing a genus-identifying region or an amplicon sequence variant (ASV) region of the microbiota nucleic acids to provide microbiota identifying information; and determining the relative abundance of the one or more genera of stool microbiota based on the microbiota identifying information.

Embodiment 11. The method of embodiment 10, wherein the method comprises sequencing an ASV region.

Embodiment 12. The method of embodiment 11, wherein the ASV region comprises a V4-V5 region of 16S rRNA gene.

Embodiment 13. The method of any of the foregoing embodiments, wherein the patient is 1 week old to 6 months old, alternatively 1 week old to 12 months old, alternatively 1 week old to three years old, alternatively 1 week old to four years old.

Embodiment 14. The method of any of the foregoing embodiments, wherein the therapeutic intervention reduces a risk of advanced stage lung disease for the patient or a risk of the patient requiring a lung transplant.

Embodiment 15. The method of any of the foregoing embodiments, wherein the therapeutic intervention comprises altering an intestinal microbiome of the patient.

Embodiment 16. The method of any of the foregoing embodiments, wherein the therapeutic intervention comprises (i) at least one pancreatic enzyme replacement product; (ii) at least one probiotic; (iii) at least one prebiotic; (iv) at least one antibiotic; (v) at least one anti-inflammatory medication; (vi) at least one mucus-thinning drug; (vii) at least one cystic fibrosis transmembrane conductance regulator (CFTR) function-improving medication; or (viii) at least one bronchodilator or inhaled medication.

Embodiment 17. The method of any of the foregoing embodiments, wherein the therapeutic intervention reduces a risk of upper respiratory infection selected from the group consisting of Staphylococcus aureus infection, Pseudomonas aeruginosa infection, Stenotrophomonas spp. infection, Streptococcus spp. infection, Haemophilus influenzae infection, nontuberculous Mycobacterium infection, Burkholderia cepacia complex infection, viral infection, and a co-infection with multiple pathogens.

Embodiment 18. A computer-implemented system for identifying a patient with cystic fibrosis as having a high risk for upper respiratory infections based on the patient's stool microbiota, comprising a machine learning model trained with data comprising: (a) risk classifications for subjects with cystic fibrosis (CF), wherein the subjects are classified as having: (i) a high risk of upper respiratory infections (URIs), or (ii) a low risk of URIs and/or a medium risk of URIs; (b) stool microbiota information for each of the subjects; wherein the trained machine learning model is configured to analyze a patient's stool microbiota information as input values, and to provide the patient's risk classification as an output value.

Embodiment 19. The computer-implemented system of embodiment 18, wherein the subject is classified as high risk when the subject has a URI frequency of more than 1 per year.

Embodiment 20. The computer-implemented system of any of embodiments 18-19, wherein the patient's risk classification is determined based upon a relative abundance of said one or more genera of stool microbiota.

Embodiment 21. The computer-implemented system of any of embodiments 18-20, wherein the trained machine learning model is configured to receive microbiota sequence data indicating relative abundance of genera or of genus-identifying regions in the patient's stool microbiota as input values.

Embodiment 22. The computer-implemented system of embodiment 21, wherein the microbiota sequence data comprises amplicon sequence variant (ASV) region sequence data.

Embodiment 23. The computer-implemented system of any of embodiments 18-21, wherein said one or more genera or ASV regions of stool microbiota comprises Faecalibacterium, Butyricoccus, or Bacteroides.

Embodiment 24. The computer-implemented system of any of embodiments 18-23, wherein the patient is predicted as being at high risk of having more than one URI per year.

Embodiment 25. The computer-implemented system of any of embodiments 18-24, wherein the computer-implemented system comprises a processor and a memory medium which stores a plurality of instructions which, when executed by the processor, cause the processor to apply the trained machine learning model to the patient's stool microbiota data and to provide the patient's category of URI risk.

Embodiment 26. A computer-implemented system for identifying a patient with cystic fibrosis as having systemic inflammation based on the patient's stool microbiota, comprising a machine learning model trained with data comprising: (a) risk classifications for subjects with cystic fibrosis (CF), wherein the subjects are classified as having: (i) high systemic inflammation, or (ii) medium systemic inflammation and/or low systemic inflammation; (b) stool microbiota information for each of the subjects; wherein the trained machine learning model is configured to analyze a patient's stool microbiota information as input values, and to provide the patient's systemic inflammation as an output value.

Embodiment 27. The computer-implemented system of embodiment 26, wherein the patient's risk classification is determined based upon a relative abundance of said one or more genera of stool microbiota.

Embodiment 28. The computer-implemented system of embodiment 26 or 27, wherein the trained machine learning model is configured to receive microbiota sequence data indicating relative abundance of genera or of genus-identifying or amplicon sequence variant (ASV) regions in the patient's stool microbiota as input values.

Embodiment 29. The computer-implemented system of embodiment 28, wherein the microbiota sequence data comprises ASV region sequence data.

Embodiment 30. The computer-implemented system of embodiment 28, wherein said one or more genera or ASV regions of stool microbiota comprises Lactococcus, Kluyvera, or Klebsiella.

Embodiment 31. The computer-implemented system of any of embodiments 26-30, wherein the subjects are classified based on neutrophil-to-lymphocyte ratio (NLR) as a biomarker.

Embodiment 32. The computer-implemented system of any of embodiments 26-31, wherein the computer-implemented system comprises a processor and a memory medium which stores a plurality of instructions which, when executed by the processor, cause the processor to apply the trained machine learning model to the patient's stool microbiota data and to provide the patient's category of systemic inflammation.

Embodiment 33. The computer-implemented system of any of embodiments 18-32, wherein the machine learning model is a random forest model.

What is claimed is:

1. A method of reducing frequency and/or number of upper respiratory infections in a patient with cystic fibrosis, the method comprising:
identifying a patient with cystic fibrosis (CF) as having a high risk for upper respiratory infections (URIs) based on the patient's stool microbiota, and
providing a therapeutic intervention to the patient.

2. The method of claim 1, wherein the patient is identified as having a high risk for frequent URIs.

3. The method of claim 1, wherein the patient is identified as having a high risk for numerous URIs.

4. The method of claim 1, wherein the high risk is determined based upon a relative abundance of one or more genera of stool microbiota.

5. The method of claim 1, wherein said one or more genera of stool microbiota comprises Faecalibacterium, Butyricoccus, or Bacteroides.

6. The method of claim 1, wherein the patient is predicted as being at high risk of having more than one URI per year.

7. The method of claim 1, wherein the method further comprises:
collecting a stool sample from the patient;
extracting microbiota nucleic acids from the stool sample;
sequencing a genus-identifying region or an amplicon sequence variant (ASV) region of the microbiota nucleic acids to provide microbiota identifying information; and
determining the relative abundance of the one or more genera of stool microbiota based on the microbiota identifying information.

8. The method of claim 7, wherein the method comprises sequencing an ASV region.

9. The method of claim 8, wherein the ASV region comprises a V4-V5 region of 16S rRNA gene.

10. The method of claim 1, wherein the patient is 1 week old to 6 months old, alternatively 1 week old to 12 months old, alternatively 1 week old to three years old, alternatively 1 week old to four years old.

11. The method of claim 1, wherein the therapeutic intervention reduces a risk of advanced stage lung disease for the patient or a risk of the patient requiring a lung transplant.

12. The method of claim 1, wherein the therapeutic intervention comprises altering an intestinal microbiome of the patient.

13. The method of claim 1, wherein the therapeutic intervention comprises (i) at least one pancreatic enzyme replacement product; (ii) at least one probiotic; (iii) at least one prebiotic; (iv) at least one antibiotic; (v) at least one anti-inflammatory medication; (vi) at least one mucus-thinning drug; (vii) at least one cystic fibrosis transmembrane conductance regulator (CFTR) function-improving medication; or (viii) at least one bronchodilator or inhaled medication.

14. The method of claim 1, wherein the therapeutic intervention reduces a risk of upper respiratory infection selected from the group consisting of Staphylococcus aureus infection, Pseudomonas aeruginosa infection, Stenotrophomonas spp. infection, Streptococcus spp. infection, Haemophilus influenzae infection, nontuberculous Mycobacterium infection, Burkholderia cepacia complex infection, viral infection, and a co-infection with multiple pathogens.

15. A computer-implemented system for identifying a patient with cystic fibrosis as having a high risk for upper respiratory infections based on the patient's stool microbiota, comprising a machine learning model trained with data comprising:
(a) risk classifications for subjects with cystic fibrosis (CF), wherein the subjects are classified as having:
(i) a high risk of upper respiratory infections (URIs), or
(ii) a low risk of URIs and/or a medium risk of URIs;
(b) stool microbiota information for each of the subjects;
wherein the trained machine learning model is configured to analyze a patient's stool microbiota information as input values, and to provide the patient's risk classification as an output value.

16. The computer-implemented system of claim 15, wherein the subject is classified as high risk when the subject has a URI frequency of more than 1 per year.

17. The computer-implemented system of claim 15, wherein the patient's risk classification is determined based upon a relative abundance of said one or more genera of stool microbiota.

18. The computer-implemented system of claim 15, wherein the trained machine learning model is configured to receive microbiota sequence data indicating relative abundance of genera or of genus-identifying regions in the patient's stool microbiota as input values.

19. The computer-implemented system of claim 15, wherein said one or more genera or ASV regions of stool microbiota comprises Faecalibacterium, Butyricoccus, or Bacteroides.

20. The computer-implemented system of claim 15, wherein the computer-implemented system comprises a processor and a memory medium which stores a plurality of instructions which, when executed by the processor, cause the processor to apply the trained machine learning model to the patient's stool microbiota data and to provide the patient's category of URI risk.
